(19) United States
(12) Patent Application Publication
(10) Pub. No.: US 2011/0190247 A1
(43) Pub. Date: Aug. 4, 2011

(54) CYCLOPROPYLCHROMENE DERIVATIVES AS MODULATORS OF THE ALPHA-2C RECEPTOR

(75) Inventors: Younong Yu, East Brunswick, NJ (US); Manuel De Lera Ruiz, Branchburg, NJ (US); Christopher W. Boyce, Flemington, NJ (US); Kevin D. McCormick, Basking Ridge, NJ (US)

(73) Assignee: Schering Corporation, Kenilworth, NJ (US)

(21) Appl. No.: 13/057,376

(22) PCT Filed: Aug. 3, 2009
(86) PCT No.: PCT/US2009/052541

§ 371 Date: Apr. 20, 2011

Related U.S. Application Data
(60) Provisional application No. 61/085,925, filed on Aug. 4, 2008.

(51) Int. Cl. A61K 31/422; A61K 31/56; A61K 31/506; A61K 31/4178; C07D 405/10; C07D 413/14; C07D 405/08; A61P 25/24; A61P 11/02; A61P 29/00; A61P 25/28; A61P 25/18; A61P 25/06; A61P 25/22; A61P 27/06; A61P 9/10; A61P 9/04; A61P 3/10

(52) U.S. Cl. 514/171; 514/256; 514/378; 514/397; 544/328; 544/333; 548/247; 548/311.4

(57) ABSTRACT

In its many embodiments, the present invention provides a novel class of cyclopropylchromene derivatives as modulators of a2C adrenergic receptor, methods of preparing such compounds, pharmaceutical compositions containing one or more such compounds, methods of preparing pharmaceutical formulations comprising one or more such compounds, and methods of treatment, prevention, inhibition, or amelioration of one or more conditions associated with the a2C adrenergic receptors using such compounds or pharmaceutical compositions.

### RELATED APPLICATIONS

This application claims benefit of U.S. provisional application U.S. Ser. No. 61/085,925 filed on Aug. 4, 2008, herein incorporated by reference.

### FIELD OF THE INVENTION

The present invention relates to compounds useful as alpha-2C (or “α2C”) adrenergic receptor modulators, methods for making these compounds, pharmaceutical compositions containing the compounds, and methods of treatment and prevention using the compounds and compositions to treat disease states associated with the modulation of the alpha-2C receptor, such as congestion (including nasal), migraine, congestive heart failure, cardiac ischemia, glaucoma, stress-induced urinary incontinence, Alzheimer's disease, Parkinson's disease, attention deficit hyperactivity disorder, pain and psychotic disorders (e.g., depression and schizophrenia).

### BACKGROUND OF THE INVENTION

The initial classification of adrenergic receptors into α- and β-families was first described by Ahlquist in 1948 (Ahlquist R P, “A Study of the Adrenergic Receptors,” Am. J. Physiol. 153:586-600 (1948)). Functionally, the α-adrenergic receptors were shown to be associated with most of the excitatory functions (vasoconstriction, stimulation of the uterus and pupil dilation). β-adrenergic receptors were implicated in vasodilation, bronchodilation and myocardial stimulation (Lands et al., “Differentiation of Receptor Systems Activated by Sympathomimetic amines,” Nature 214:597-598 (1967)). Since this early work, α-adrenergic receptors have been subdivided into α1- and α2-adrenergic receptors. Cloning and expression of α-adrenergic receptors have confirmed the presence of multiple subtypes of both α1-(α1A, α1B, α1D) and α2-(α2A, α2B, α2C) adrenergic receptors (Michel et al., “Classification of α₁-Adrenoceptor Subtypes,” Naunyn-Schmiedeberg's Arch. Pharmacol, 352:1-10 (1995); Macdonald et al., “Gene Targeting—Homing in on α₂-Adrenoceptor-Subtype Function,” TIPS, 18:211-219 (1997)).

Current therapeutic uses of α-2 adrenergic receptor drugs involve the ability of those drugs to mediate many of the physiological actions of the endogenous catecholamines. There are many drugs that act on these receptors to control hypertension, intraocular pressure, eye reddening and nasal congestion and induce analgesia and anesthesia.

α2 adrenergic receptors can be found in the rostral ventrolateral medulla, and are known to respond to the neurotransmitter norepinephrine and the antihypertensive drug clonidine to decrease sympathetic outflow and reduce arterial blood pressure (Bousquet et al., “Role of the Ventral Surface of the Brain Stem in the Hypothesive Action of Clonidine,” Eur. J. Pharmacol., 34:151-156 (1975); Bousquet et al., “Imidazoline Receptors: From Basic Concepts to Recent Developments,” 26:S1-S6 (1995)). Clonidine and other imidazolines also bind to imidazoline receptors (formerly called imidazoline-guanidinium receptive sites or IGRS) (Bousquet et al., “Imidazoline Receptors: From Basic Concepts to Recent Developments,” 26:S1-S6 (1995)). Some researchers have speculated that the central and peripheral effects of imidazolines as hypotensive agents may be related to imidazoline receptors (Bousquet et al., “Imidazoline Receptors: From Basic Concepts to Recent Developments,” 26:S1-S6 (1995); Reis et al., “The Imidazoline Receptor: Pharmacology, Functions, Ligands, and Relevance to Biology and Medicine,” Ann. N.Y. Acad. Sci., 763:1-703 (1995). Compounds having adrenergic activity are well-known in the art and are described in numerous patents and scientific publications. It is generally known that adrenergic activity is useful for treating animals of the mammalian species, including humans, for curing or alleviating the symptoms and conditions of numerous diseases and conditions. In other words, it is generally accepted in the art that pharmaceutical compositions having an adrenergic compound or compounds as the active ingredient are useful for treating, among other things, glaucoma, chronic pain, migraines, heart failure, Alzheimer's disease, attention deficit hyperactivity disorder, Parkinson's disease and psychotic disorders (e.g., schizophrenia and depression).

For example, published PCT application WO 02/076950 discloses compounds having α2 agonist activity of the following general formula:

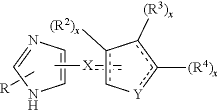

Other publications disclosing similar compounds include WO 01/00586, WO 99/28300, U.S. Pat. No. 6,841,684 B2 and US 2003/0023098 A1.

A class of compounds having α2-agonist properties is disclosed in U.S. Pat. No. 5,658,938, and has the following general formula:

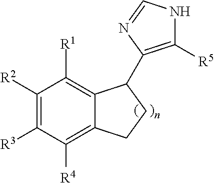

wherein n=1-2, R¹-R³ represent hydrogen, halogen hydroxy, alkyl or alkoxy, and R⁵ is hydrogen or alkyl.

Another class of compounds reported to have affinity for α2 receptors includes the following two compounds (Bagley et. al., Med. Chem. Res. 1994, 4:346-364):

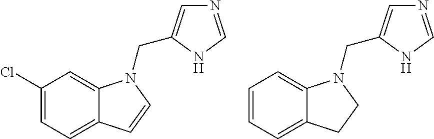

It is also known that compounds having adrenergic activity, such as α2A agonists, may be associated with undesirable side effects. Examples of such side effects include hyper- and hypotension, sedation, locomotor activity, psychotic disorders (e.g., schizophrenia).

Another class of compounds reported to have affinity for α2 receptors includes the following two compounds (Miller et. al., J. Med. Chem. 1994, 37:2328-2333; J. Med. Chem. 1996, 39:3001-3013; J. Med. Chem. 1997, 37:3014-3024):

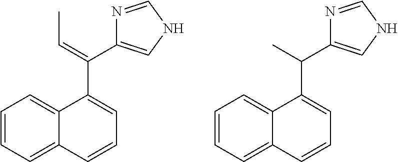

Another class of indane and tetrahyrdonaphthalene type compounds having α2-agonist properties is disclosed in PCT application WO 97/12874 and WO20040506356.

This class has the following general formula:

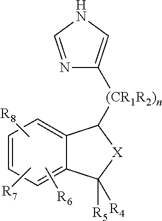

wherein n=0-1, X is 1 or 2 carbon units, R₄ is H, OH, alkyl, or alkoxy, R₅ may be taken together with R⁴ to form a carbonyl, and R⁶-R⁸═H, OH, SH, alkyl, alkenyl, cycloalkyl, alkoxy, hydroxyalkyl, alkylthio, alkylthiol, halo, CF₃, NO₂, or alkylamino. This class specifically includes MPV-2426 (fadolmidine) and its prodrug esters:

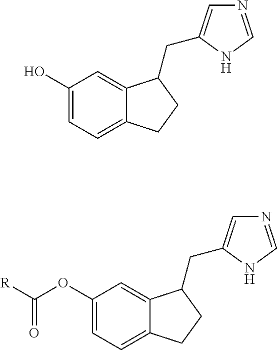

wherein R is optionally substituted lower alkyl, aryl, cycloalkyl, heteroaryl, lower alkylamino, and saturated 5- or 6-membered heterocyclic groups containing 1 or 2 N atoms.

WO2008/052907 discloses substituted 2-imidazoles of the formula

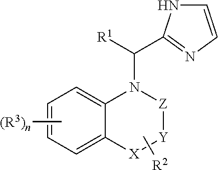

These compounds are said to act as modulators of TRACE amine associated receptors.

Further, other classes of compounds that exhibit activity functional selectivity for the alpha-2C receptor have been discovered. Application U.S. Ser. No. 11/508,458, filed Aug. 23, 2006, discloses indoline compounds that possess this activity and application U.S. Ser. No. 11/508,467, filed on the same date, describes morpholine compounds that are active or functionally selective of the alpha-2C receptor. CIP applications of these applications have been filed; the Ser. Nos. 11/705,673 and 11/705,683, both filed on Feb. 13, 2007.

Additional published co-pending applications that disclose alpha2C receptor agonists include WO2008/100456 (AL06619), WO2008/100459 (AL06620), WO2008/100480 (AL06621) and WO/2009/020578 (AL06693).

Compounds that act as antagonists of the alpha-2C receptor are also known in the art. Hoeglund et al. describe quinoline derivatives that are said to be potent and selective alpha 2C antagonists and said to be useful in treating “certain psychiatric disorders such as depression and schizophrenia” (Hoeglund et al., J. Med. Chem. 49:6351-6363 (2006)). WO 2001/64645 to Orion Corp. also describes quinoline derivatives that are alpha-2C recptor antagonists and indicates that these compounds are useful for the treatment of conditions of the pheripheric or CNS system, including treating depression, anxiety, post traumatic stress disorder, schizophrenia, Parkinson's disease and other movement disorders, and dementias (e.g., Alzheimer's disease). WO 2003/082825, also to Orion Corp., indicates alpha-2C receptor antagonists have utility in treating symptoms of disorders and conditions with sensorimotor-gating deficits. Selliner et al., indicate that acridin-9-yl-[4-(4-methylpiperazinal-1-yl)-phenyl]amine is a highly selective alpha-2C adrenergic receptor antagonist and may be useful intreating neuropsychiatric disorders (Salliner et al., British J. Pharmacol. 150:391-402 (2007)).

It is also known that compounds having adrenergic activity, such as α2A agonists, may be associated with undesirable side effects. Examples of such side effects include hyper- and hypotension, sedation, locomotor activity, and body temperature variations.

Moreover, substituted indolinone-type compounds are known in the art for treating cancer. Such compounds are described in US 2005/0090541 A1 (Berlex Biosciences) and WO 2007/008664 A1 (Allergan).

Further, imidazolylmethylenetetralone analogues are described in the art. WO 94/070866 describes imidazolylmethylenetetralone

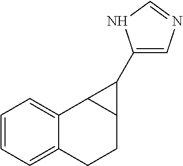

and teaches that this compound, as well as some alkoxy, nitro, and alkoxycarbonyl substituted analogues thereof, possess utility as aromatase inhibitors. Wachter et al. describes the 4-imidazole analogues as an inhibitor of steroidogenic enzymes P450 aromatase and P450 15 (Wachter et al., J. Med. Chem. 39:841-841 (1996)). Hartmann et al. describe compounds of the formula:

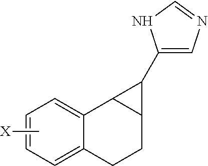

where X is H or methoxy, and indicate that these compounds will inhibit aldosterone synthase and may be used in treating congestive heart failure and myocardial fibrosis (Hartmann et al., Eur. J. of Med. Chem. 38(4):363-366 (2003)). Schappach et al., describe inhibitors of human 17α-hydroxlylase-17,20-lyase that have the following structure:

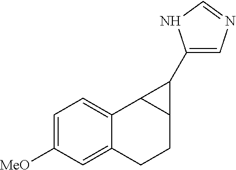

### (Schappach et al., Phaemazie 56(11):835-842 (2001).

U.S. Pat. No. 6,673,337 describes and claims an ophthalmic composition comprising an alpha-2C agonist component and a solubility enhancing component other than cyclodextrin. The patent does not specifically describe alpha-2C receptor agonists.

U.S. Pat. No. 6,127,396 describes compounds of the formula:

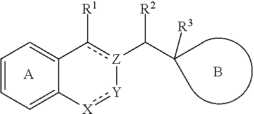

where inter alia ring A is an optionally substituted benzene ring, Z is CR⁴, and (R¹ and R⁴) or (R² and R³) can form a cyclopropyl ring. These compounds are said to exhibit α2-adrenergic activity or inhibit the re-uptake of serotonin and/or noradrenaline.

It has been discovered in accordance with the present invention that the inventive compounds act as modulators of the alpha-2C receptor (i.e., they can act as alpha-2C receptor agonists or as alpha-2C receptor antagonists) and are useful in treating disorders modulated by the alpha-2C receptor.

There is a need for new compounds, formulations, treatments and therapies to treat diseases and disorders associated with α2C adrenergic receptors. Further, there is a need for alpha-2C receptor modulators that minimize adverse side effects, such as those associated with the alpha-2A receptor subtype (viz., blood pressure or sedation). It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds that are modulators of the α2C adrenergic receptor, or metabolites, stereoisomers, salts, solvates or polymorphs thereof, methods of preparing such compounds, pharmaceutical compositions comprising one or more such compounds, methods of preparing pharmaceutical formulations comprising one or more such compounds, and methods of treatment, prevention, inhibition or amelioration of one or more conditions associated with α2C receptors using such compounds or pharmaceutical compositions.

In one aspect, the present application discloses a compound, or pharmaceutically acceptable salts or metabolites, solvates, prodrugs or polymorphs of said compound, said compound having the general structure shown in Formula I

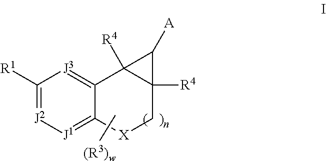

wherein:

J¹, J² and J³ are independently —N—, —N(O)—, or —C(R²)—;

X is —C(R⁶)—; —N(R¹⁴)—, —O—, or —S—;

A is a 5-membered heteroaryl, heterocyclyl or heterocyclenyl ring containing 1-3 heteroatoms, preferably selected from the group consisting of —O—, —S— and —N—, and is optionally substituted with at least one (preferably 1 to 5, more preferably 1 to 3) R⁵ and/or 1 or 2 (═O) (carbonyl) groups;

R¹ is selected from the group consisting of —[C(R^{a})(R^{b})]_{q}YR^{7′}, —[C(R^{a})(R^{b})]_{q}N(R⁷)YR^{7′}, —[C(R^{a})(R^{b})]_{q}NR⁷R^{7′}, —[C(R^{a})(R^{b})]_{q}OYR^{7′}, —[C(R^{a})(R^{b})]_{q}N(YR⁷)(YR⁷′), —[C(R^{a})(R^{b})]_{q}ON═CR⁷R^{7′} and —[C(R^{a})(R^{b})]_{q}CN;

Y is selected from the group consisting of a bond, —C(═O)—, —C(═O)NR⁷—, —C(═O)O—, —C(═O)—[C(R^{a})(R^{b})]ₙ—O—C(═O)—, —C(═O)N(R^{c})—O—, —C(═NR⁷)—, —C(═NOIR⁷)—, —C(═NR⁷)NR⁷—, —C(═N1R⁷)NR⁷O—, —C(═N—CN)—, —S(O)ₚ—, —SO₂NR⁷—, and —C(═S)NR⁷—;
wherein R^{a} and R^{b} are independently selected from the group consisting of H, alkyl, alkoxy, and halo, and
R^{b} is H or alkyl;

R² is independently selected from the group consisting of H, —OH, halo, —CN, —NO₂, —S(O)ₚR⁷, —NR⁷R^{7′}, —[C(R^{a})(R^{b})]ₚYR^{7′}, —[C(R^{a})(R^{b})]ₚN(R⁷)YR^{7′}, —[C(R^{a})(R^{b})])ₚ═OYR^{7′}, and —(CH₂)ₚON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R³ is independently selected from the group consisting of H, halo, and (═O), and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one (preferably 1 to 5, more preferably 1 to 3) R⁵, provided that when w is 3, no more than 2 of the R³ groups may be (═O);

R⁴ is independently selected from the group consisting of H, —CN, and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one (preferably 1 to 5, more preferably 1 to 3) R⁵;

R⁵ is independently selected from the group consisting of H, halo, —OH, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one (preferably 1 to 5, more preferably 1 to 3) of halo, —OH, —CN, —NO₂, —NR⁷R^{7′}, and —S(O)ₚR⁷ substituents and/or 1 or 2 (═O);

R⁶ is independently selected from the group consisting of H —CN, and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one (preferably 1 to 5, more preferably 1 to 3) of halo, —OH, —CN, —NO₂, —NR⁷R^{7′}, and —S(O)ₚR⁷ substituents and/or 1 or 2 (═O), and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times (preferably 1 to 5, more preferably 1 to 3) by R¹²;

R^{7′} is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times (preferably 1 to 5, more preferably 1 to 3) by R¹²; or
a) when a variable is —NR⁷R^{7′}, —[C(R^{a})(R^{b})]_{q}YR^{7′}, —C[(R^{a})(R^{b})]_{q}NR⁷R^{7′}, [C(R^{a})(R^{b})]_{q}OYR^{7′}, —(CH₂)_{c}, NR⁷R^{7′}, —C(O)NR⁷R^{7′} or —SO₂NR⁷R^{7′}, R⁷ and R^{7′} together with the nitrogen atom to which they are attached independently form a 3- to 8-membered heterocyclyl, heterocyclenyl or heteroaryl ring having, in addition to the N atom, 1 or 2 additional hetero atoms independently selected from the group consisting of O, N, —N(R⁹)— and S, wherein said rings are optionally substituted by 1 to 5 independently selected R⁵ moieties and/or 1 or 2 (═O), or
b) when a variable is —(CH₂)_{q}ON═CR⁷R^{7′} or —[C(R^{a})(R^{b})]_{q}ON═CR⁷R^{7′}, R⁷ and R^{7′} together with the carbon atom to which they are attached independently form a 3- to 8-membered cycloalkyl, cycloalkenyl, aryl, heterocyclyl, heterocyclenyl or heteroaryl ring, wherein said hetroacyclyl, heterocyclenyl or heteroaryl rings have 1-3 heteroatoms which are independently selected from the group consisting of O, N, —N(R⁹)— and S, wherein said rings are optionally substituted by 1 to 5 independently selected R⁵ moieties and/or 1 or 2 (═O); R⁹ is independently selected from the group consisting of H, —C(O)—R¹⁰, —C(O)—OR¹⁰, and —S(O)ₚ—OR¹⁹ and alkyl, alkenyl, alkynyl, cycloalkyl, aryl, arylalkyl, heteroaryl, and heteroarylalkyl groups, each of which is optionally substituted with at least one (preferably 1 to 5, more preferably 1 to 3) of halo, —OH, —CN, —NO₂, —N(R¹¹)₂, and —S(O)ₚR¹¹ substituents and/or 1 or 2 (═O);

R¹⁰ is selected from the group consisting of alkyl, alkenyl, alkynyl, cycloalkyl, aryl, arylalkyl, heteroaryl, and heteroarylalkyl groups, each of which is optionally substituted with at least one (preferably 1 to 5, more preferably 1 to 3) of halo, —OH, —CN, —NO₂, —N(R¹¹)₂, and —S(O)ₚR¹¹ substituents and/or 1 or 2 (═O);

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl, each of which is optionally substituted by at least one (preferably 1 to 5, more preferably 1 to 3) substituent independently selected from the group consisting of halo, —OH, —CN, —NO₂, —N(R^{11′})₂, and —S(O)ₚR¹¹ substituents and/or 1 or 2 (═O);

R^{11′} is independently selected from the group consisting of H, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

R¹² is independently selected from the group consisting of H, halo, —OH, —CN, —NO₂, —N(R¹¹)₂, and —S(O)ₚR¹¹, and/or 1 or 2 (═O), and alkyl, alkoxy, alkenyl, alkenyloxy, alkynyl, cycloalkyl, cycloalkenyl, cycloalkoxy, aryl, aryloxy, arylalkyl, heteroaryl, heteroaryloxy, heteroarylalkyl, heterocyclyl, heterocyclenyl, heterocyclenyloxy, heterocyclylalkyl, heterocyclenylalkyl, arylalkoxy, heteroarylalkoxy, heterocyclylalkoxy, and heterocyclenylalkoxy groups, each of which in turn is optionally substituted by at least once (preferably 1 to 5, more preferably 1 to 3) by a substituent selected from the group consisting of H, alkyl, haloalkyl, halo, —OH, optionally substituted alkoxy, optionally substituted aryloxy, optionally substituted cycloalkoxy, optionally substituted heteroaryloxy, optionally substituted heterocyclenyloxy, —CN, —NO₂, —N(R¹¹)₂, and —S(O)ₚR¹¹ and/or 1 or 2 (═O), wherein said optionally substituted alkoxy, aryloxy, optionally substituted cycloalkoxy, optionally substituted heteroaryloxy, and heterocyclenyloxy when substituted are substituted one or more (preferably 1 to 5, more preferably 1 to 3) times by R¹¹;

R¹⁴ is selected from the group consisting of H, —C(O)—R¹⁰, and —S(O)ₚOR¹⁰ and alkyl, alkenyl, alkynyl, cycloalkyl, aryl, arylalkyl, heteroaryl, and heteroarylalkyl groups, each of which is optionally substituted with at least one (preferably 1 to 5, more preferably 1 to 3) halo, —OH, —CN, —NO₂, —N(R¹¹)₂, and —S(O)ₚR¹¹ and/or 1 or 2 (═O);

n is independently 0, 1, 2 or 3;

w is 0, 1, 2, or 3;

p is independently 0, 1 or 2; and

q is independently an integer from 0-10;
provided that when
(a) X=—C(R²)—, R²=H, and R¹=—[C(R^{a})(R^{b})]_{q}YR^{7′}, q=0, and Y=—C(═O)O—, then R^{7′} cannot be H or alkyl;
(b) X=—C(R²)—, R²=H, and R¹=—[C(R^{a})(R^{b})]_{q}OYR^{7′}, q=0, and Y is a bond, then R^{7′} cannot be H or alkyl; and
(c) X=—C(R²)—, R²=H, and R¹=—[C(R^{a})(R^{b})]_{q}YR^{7′}, q=0, and Y is a bond, R^{7′} cannot be H.

The compounds of Formula I can be useful as α2C adrenergic receptor modulators and can be useful in the treatment or prevention of one or more conditions associated with the α2C receptor by administering at least one compound of Formula I to a mammal in need of such treatment. Conditions that may be treated by modulating the α2C receptor include allergic rhinitis, congestion (including congestion associated with perennial allergic rhinitis, seasonal allergic rhinitis, non-allergic rhinitis, vasomotor rhinitis, rhinitis medicamentosa, sinusitis, acute rhinosinusitis, or chronic rhinosinusitis, congestion caused by polyps, or caused by the common cold), pain (e.g., neuropathy, inflammation, arthritis, or diabetis), diarrhea, glaucoma, congestive heart failure, chronic heart failure, cardiac ischemia, manic disorders, depression, anxiety, migraine, stress-induced urinary incontinence, neuronal damage from ischemia, schizophrenia, attention deficit hyperactivity disorder, symptoms of diabetes, post traumatic stress disorder, Parkinson's disease or a dementia (e.g., Alzheimer's disease).

Another embodiment of this invention is the treatment or prevention of one or more conditions associated with the α2C receptor by administering at least one compound of Formula I to a mammal in need of such treatment by selectively modulating α2C adrenergic receptors in the mammal.

Another embodiment of this invention is the treatment or prevention of one or more conditions associated with the α2C receptor by administering an effective amount at least one compound of Formula I to a mammal in need of such treatment without modifying blood pressure at the therapeutic dose.

Another embodiment of the present invention is a method for selectively modulating α2C adrenergic receptors in a cell in a mammal in need thereof, comprising contacting said cell with a therapeutically effective amount of at least one compound of Formula I or a pharmaceutically acceptable salt, ester, prodrug or salt thereof.

Another embodiment of the present invention is a method for the treatment of congestion in a mammal in need thereof without modifying the blood pressure at therapeutic doses which comprises administering to the mammal an effective dose of at least one compound having adrenergic activity wherein said compound is a selective agonist of the α2C receptor.

### DETAILED DESCRIPTION

In an embodiment, the present invention discloses certain cyclopropyl-chromene compounds which are represented by structural Formula I, or a pharmaceutically acceptable salt or solvate thereof, wherein the various moieties are as described above.

In another embodiment, J¹, J² and J³ are each —C(R²)—.

In another embodiment, J¹, J² and J³ are each —CH—.

In another embodiment, J¹, J² and J³ are each —N—.

In another embodiment, J¹, J² and J³ are independently —CR²— or —N—.

In another embodiment, J¹ and J² are —CH— and J³ is —N—.

In another embodiment, A is unsubstituted imidazole.

In another embodiment, n is 0.

In another embodiment, n is 1.

In another embodiment, n is 2.

In another embodiment, n is 3.

In another embodiment, p is 1 or 2.

In another embodiment, q is an from integer 0-3.

In another embodiment, X is —CH₂—.

In another embodiment, X is —NH— or —N(alkyl)-.

In another embodiment, X is —O—.

In another embodiment, X is —S—.

In another embodiment, J¹, J² and J³ are —CH— is —N— and X is —CH₂—.

In another embodiment, J¹, J² and J³ are —CH— and J³ is —N— and X is —NH— or —N(alkyl)-.

In another embodiment, J¹, J² and J³ are —CH— and J³ is —N— and X is —O—.

In another embodiment, J¹, J² and J³ are —CH— and J³ is —N— and X is —S—.

In another embodiment, J¹, J² and J³ are —CH— and J³ is —N— and X is —NH— or —N(alkyl)—,

In another embodiment, J¹, J² and J³ are —CH— and J³ is —N— and X is —O—.

In another embodiment, J¹, J² and J³ are —CH— and J³ is —N— and X is —S—.

In another embodiment, R² is H, halo, —CN, alkyl (e.g., methyl, ethyl, propyl, butyl, sec-butyl, or t-butyl), cycloalkyl (e.g., cyclopropyl, cyclobutyl, cyclopentyl or cyclohexyl) or haloalkyl (e.g., trifluororomethyl).

In another embodiment J³ is N or —(R²)—, where R² is H, halo, —CN, methyl, ethyl, or trifluororomethyl.

In another embodiment R¹ is hydrogen, cyano, —N(R⁷)C(═O)OR^{7′}, —N(R⁷)C(═O)NH(R^{7′}), —N(R⁷)C(═O)R^{7′}, heteroaryl or heterocyclenyl.

In another embodiment R⁴ is H or alkyl.

In another embodiment R⁷ and R^{7′} are independently H, methyl, ethyl, propyl, cyclopropyl, wherein said methyl, ethyl, propyl or cyclopropyl groups are optionally substituted with one or more substituents independently selected from the group consisting of halogen, cyano of methoxy.

In an alternative embodiment R⁷ or R^{7′} are independently H, alkyl, haloalkyl, or an optionally substituted heteroaryl (e.g., pyrimidine, pyridine, 1,2-diazole, imidazole, thiazole, pyrazole, thienyl, or thiophenyl), which are optionally substituted one or more times (preferably once or twice) by substituents independently selected from the group consisting of alkyl, halo, haloalkyl, alkoxy, haloalkoxy, cyano, nitro, amino, alkylamino, or dialkylamino.

In another embodiment R¹³ is H or alkyl, alkenyl cycloalkyl, aryl or heteroaryl, each optionally substituted one or more times (preferably 1 or 2 times) by alkyl, halogen, haloalkyl, alkoxy, haloalkoxy, cyano, nitro or —N(R¹¹)₂— (e.g., R¹¹ is independently H or alkyl).

In an other embodiment R¹ is —[C(R^{a})(R^{b})]_{q}YR^{7′}, where Y is a bond, q is 0 or 1, and R^{7′} is aryl (e.g., phenyl) or heteroaryl, (e.g., pyrimidine, pyridine, 1,2-diazole, imidazole, thiazole, pyrazole, thienyl, or thiophenyl), which are optionally substituted one or more times (preferably once or twice) by substituents independently selected from the group consisting of alkyl, halo, haloalkyl, alkoxy, haloalkoxy, cyano, nitro, amino, alkylamino, or dialkylamino.

In another embodiment R¹ is —[C(R^{a})(R^{b})]_{c}CN, where q is 0 or 1.

Another embodiment of formula I are those compounds wherein:

A is

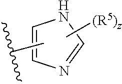

R¹ is —[C(R^{a})(R^{b})]_{q}N(R⁷)YR^{7′} or —CN,

Y is —C(═O)—, —C(═O)O— or —C(═O)NR⁷;

z is 1 or 2; and

n is 2.

In another embodiment, the present invention discloses compounds which are represented by structural formula I

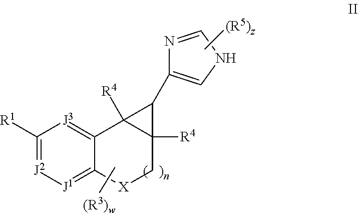

or a pharmaceutically acceptable salt, solvate or ester thereof
wherein

J¹, J² and J³ are independently —N—, or —C(R²)—;

R¹ is selected from the group consisting of H, —CN, —[C(R^{a})(R^{b})]_{q}YR^{7′}, —[C(R^{a})(R^{b}]_{q}N(R⁷)YR^{7′}, —[C(R^{a})(R^{b})]_{q}NR⁷R^{7′}, or —[C(R^{a})(R^{b})]_{q}OYR^{7′},

Y is selected from the group consisting of a bond, —C(═O)—, —C(═O)NR⁷—, —C(═O)O—, —C(═O)—[C(R^{a})(R^{b})]ₙ—O—C(═O)—, —C(═O)N(R^{c})—O—, —C(═NR⁷)—, —C(═NOR⁷)—, —C(═NR⁷)NR⁷—, —C(═NR⁷)NR⁷O—, —C(═N—CN)—, —S(O)ₚ—, —SO₂NR⁷—, and —C(═S)NR⁷—;
wherein R^{a} and R^{b} are independently selected from the group consisting of H, alkyl, alkoxy, and halo, and
R^{c} is H or alkyl;

R² is independently selected from the group consisting of H, —OH, halo, —CN, —NO₂, —S(O)ₚR⁷, —NR⁷R^{7′}, —[C(R^{a})(R^{b})]_{q}YR^{7′}, —[C(R^{a})(R^{b})]_{q}N(R⁷)YR^{7′}, —[C(R^{a})(R^{b})])═_{q}OYR^{7′}, and —(CH₂)_{q}ON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R³ is independently selected from the group consisting of H, halo, and (═O), and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵, provided that when w is 3, no more than 2 of the R³ groups may be (═O);

R⁴ is independently selected from the group consisting of H, halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R⁵ is independently selected from the group consisting of H, halo, —OH, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O);

R⁶ is independently selected from the group consisting of H and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O), and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²;

R^{7′} is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²; or
a) when a variable is —NR⁷R^{7′}, —[C(R^{a})(R^{b})]_{q}YR^{7′}, —C[(R^{a})(R^{b})]_{q}NR⁷R^{7′}, —[C(R^{a})(R^{b})]_{q}OYR^{7′}, —(CH₂)_{q}NR⁷R^{7′}, —C(O)NR⁷R^{7′} or —SO₂NR⁷R^{7′}, R⁷ and R^{7′} together with the nitrogen atom to which they are attached independently form a 3- to 8-membered heterocyclyl, heterocyclenyl or heteroaryl ring having, in addition to the N atom, 1 or 2 additional hetero atoms independently selected from the group consisting of O, N, —N(R⁹)— and S, wherein said rings are optionally substituted by 1 to 5 independently selected R⁵ moieties and/or 1 or 2 (═O), or
b) when a variable is —(CH₂)_{q}ON═CR⁷R^{7′} or —[C(R^{a})(R^{b})]_{q}ON═CR⁷R^{7′}, R⁷ and R^{7′} together with the carbon atom to which they are attached independently form a 3- to 8-membered cycloalkyl, cycloalkenyl, aryl, heterocyclyl, heterocyclenyl or heteroaryl ring, wherein said hetroacyclyl, heterocyclenyl or heteroaryl rings have 1-3 heteroatoms which are independently selected from the group consisting of O, N, —N(R⁹)— and S, wherein said rings are optionally substituted by 1 to 5 independently selected R⁵ moieties and/or 1 or 2 (═O);

R⁹ is independently selected from the group consisting of H, —C(O)—R¹⁰, —C(O)—OR¹⁰, and —S(O)ₚ—OR¹⁰ and alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O);

R¹⁰ is selected from the group consisting of alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O);

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl, each of which is optionally substituted by at least one substituent independently selected from the group consisting of halo, —OH, —CN, —NO₂, —N(R^{11′})₂, and —S(O)ₚR¹¹ substituents and/or 1 or 2 (═O);

R^{11′} is independently selected from the group consisting of H, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

R¹² is independently selected from the group consisting of H, halo, —OH, —CN, —NO₂, —N(R¹¹)₂, and —S(O)ₚR¹¹, and/or 1 or 2 (═O), and alkyl, alkoxy, alkenyl, alkenyloxy, alkynyl, cycloalkyl, cycloalkenyl, cycloalkoxy, aryl, aryloxy, arylalkyl, heteroaryl, heteroaryloxy, heteroarylalkyl, heterocyclyl, heterocyclenyl, heterocyclenyloxy, heterocyclylalkyl, heterocyclenylalkyl, arylalkoxy, heteroarylalkoxy, heterocyclylalkoxy, and heterocyclenylalkoxy groups, each of which in turn is optionally substituted by at least one by a substituent selected from the group consisting of H, alkyl, haloalkyl, halo, —OH, optionally substituted alkoxy, optionally substituted aryloxy, optionally substituted cycloalkoxy, optionally substituted heteroaryloxy, optionally substituted heterocyclenyloxy, —CN, —NO₂, —N(R¹¹)₂, and —S(O)ₚR¹¹ and/or 1 or 2 (═O), wherein said optionally substituted alkoxy, aryloxy, optionally substituted cycloalkoxy, optionally substituted heteroaryloxy, and heterocyclenyloxy when substituted are substituted one or more times by R¹¹;

R¹³ is independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R¹⁴ is selected from the group consisting of H, —C(O)—R¹⁰, and —S(O)ₚOR¹⁰ and alkyl, alkenyl, alkynyl, cycloalkyl, aryl, arylalkyl, heteroaryl, and heteroarylalkyl groups, each of which is optionally substituted with at least one halo, —OH, —CN, —NO₂, —N(R¹¹)₂, and —S(O)ₚR¹¹ and/or 1 or 2 (═O);

n is independently 0, 1, 2 or 3;

w is 0, 1, 2, 3, or 4;

p is independently 0, 1 or 2;

q is independently an integer from 0-10; and

z is 0, 1 or 2;

In another embodiment of Formula II, J¹, J² and J³ are each —C(R²)—.

In another embodiment of Formula II, J¹, J² and J³ are each —CH—.

In another embodiment of Formula II, J¹, J² and J³ are each —N—.

In another embodiment of Formula II, J¹, J² and J³ are independently —CR²— or —N—.

In another embodiment, R² is H, halo, —CN, alkyl (e.g., methyl, ethyl, propyl, butyl, sec-butyl, or t-butyl), cycloalkyl (e.g., cyclopropyl, cyclobutyl, cyclopentyl or cyclohexyl) or haloalkyl (e.g., trifluororomethyl).

In another embodiment of Formula II, J¹ and J² are —CH— and J³ is —N—.

In another embodiment of Formula II, A is unsubstituted imidazole.

In another embodiment of Formula II, n is 1.

In another embodiment of Formula II, n is 1.

In another embodiment of Formula II, n is 2.

In another embodiment of Formula II, n is 3.

In another embodiment of Formula II, p is 1 or 2.

In another embodiment of formula II, q is an integer from 0-3.

In another embodiment of formula II, w is 0, 1, or 2.

In another embodiment of formula II, z is 0, 1, or 2.

In another embodiment of Formula II, X is —CH₂—.

In another embodiment of Formula II, X is —NH— or —N(alkyl)-.

In another embodiment of Formula II, X is —O—.

In another embodiment of Formula II, X is —S—.

In another embodiment of Formula II, J¹ and J² are —CH— and J³ is —N— and X is —CH₂—.

In another embodiment of Formula II, J¹ and J² are —CH— and J³ is —N— and X is —NH— or —N(alkyl).

In another embodiment of Formula II, J¹ and J² are —CH— and J³ is —N— and X is —CH₂—.

In another embodiment of Formula II, J¹, J² and J³ are —CH— and X is —NH— or —N(alkyl).

In another embodiment of Formula II, J¹, J² and J³ are —CH— and X is —O—.

In another embodiment of Formula II, J¹, J² and J³ are —CH— and X is —S—.

In another embodiment of Formula II, J³ is N or —C(R²)—, where R² is H, halo, —CN, methyl, ethyl, or trifluororomethyl.

In another embodiment of Formula II R⁴ is independently H or alkyl.

In another embodiment of Formula II R¹⁴ is independently H or alkyl.

In another embodiment of Formula II, R¹ is hydrogen, cyano, —N(R⁷)C(═O)OR^{7′}, —N(R⁷)C(═O)NH(R^{7′}), —N(R⁷)C(═O)R^{7′}, optionally substituted aryl (e.g., phenyl), optionally substituted heteroaryl (e.g., pyrimidine, pyridine, 1,2-diazole, imidazole, thiazole, pyrazole, thienyl, or thiophenyl), optionally substituted heterocyclenyl (e.g., morpholine, pyrazine, or piperazine), wherein said optionally substituted groups are optionally substituted one or more times (preferably one or twice) by substituents independently selected from the group consisting of alkyl, halo, haloalkyl, alkoxy, haloalkoxy, cyano, nitro, amino, alkylamino, or dialkylamino.

In another embodiment of Formula II, R⁷ and R^{7′} are independently H, methyl, ethyl, propyl, cyclopropyl, wherein said methyl, ethyl, propyl or cyclopropyl groups are optionally substituted with one or more substituents selected from the group consisting of halogen, cyano of methoxy.

In another embodiment, the present invention discloses compounds which are represented by structural formula III

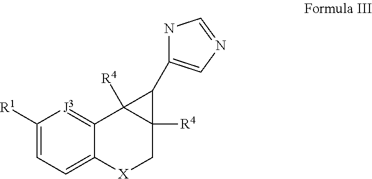

or a pharmaceutically acceptable salt, solvate or ester thereof.

In another embodiment of Formula III, J³ is —N— and X is —C(R²)—.

In another embodiment of Formula III, J³ is —N— and X is —NH— or —N(alkyl)-.

In another embodiment of Formula III, J³ is —N— and X is —O—.

In another embodiment of Formula III, J³ is —N— and X is —S—.

In another embodiment of Formula III, J³ is —CH— and X is —C(R²)—.

In another embodiment of Formula III, J³ is —CH— and X is —NH— or —N(alkyl)-.

In another embodiment of Formula III, J³ is —CH— and X is —O—.

In another embodiment of Formula III, J³ is —CH— and X is —S—.

In another embodiment of Formula III, R¹ is hydrogen, cyano, —N(R⁷)C(═O)OR^{7′}, —N(R⁷)C(═O)NH(R^{7′}), —N(R⁷)C(═O)R^{7′}, optionally substituted aryl (e.g., phenyl), optionally substituted heteroaryl (e.g., pyrimidine, pyridine, 1,2-diazole, imidazole, thiazole, pyrazole, thienyl, or thiophenyl), optionally substituted heterocyclenyl (e.g., morpholine, pyrazine, or piperazine), wherein said optionally substituted groups are optionally substituted one or more times (preferably one or twice) by substituents independently selected from the group consisting of alkyl, halo, haloalkyl, alkoxy, haloalkoxy, cyano, nitro, amino, alkylamino, or dialkylamino.

In another embodiment of Formula III, R⁷ and R^{7′} are independently H, methyl, ethyl, propyl, cyclopropyl, wherein said methyl, ethyl, propyl or cyclopropyl groups are optionally substituted with one or more substituents selected from the group consisting of halogen, cyano of methoxy.

In another embodiment of Formula III, R⁴ is H.

In another embodiment of Formula III R⁴ is independently H or methyl.

In another embodiment of Formula III, R¹ is hydrogen, cyano, —N(R⁷)C(═O)OR^{7′}, —N(R⁷)C(═O)NH(R^{7′}), —N(R⁷)C(═O)R^{7′}, optionally substituted aryl (e.g., phenyl), optionally substituted heteroaryl (e.g., pyrimidine, pyridine, 1,2-diazole, imidazole, thiazole, pyrazole, thienyl, or thiophenyl), optionally substituted heterocyclenyl (e.g., morpholine, pyrazine, or piperazine), wherein said optionally substituted groups are optionally substituted one or more times (preferably one or twice) by substituents independently selected from the group consisting of alkyl, halo, haloalkyl, alkoxy, haloalkoxy, cyano, nitro, amino, alkylamino, or dialkylamino.

In another embodiment of Formula III, R⁷ and R^{7′} are independently H, methyl, ethyl, propyl, cyclopropyl, wherein said methyl, ethyl, propyl or cyclopropyl groups are optionally substituted with one or more substituents selected from the group consisting of halogen, cyano of methoxy.

A group of compounds falling within Formula I are those shown below:

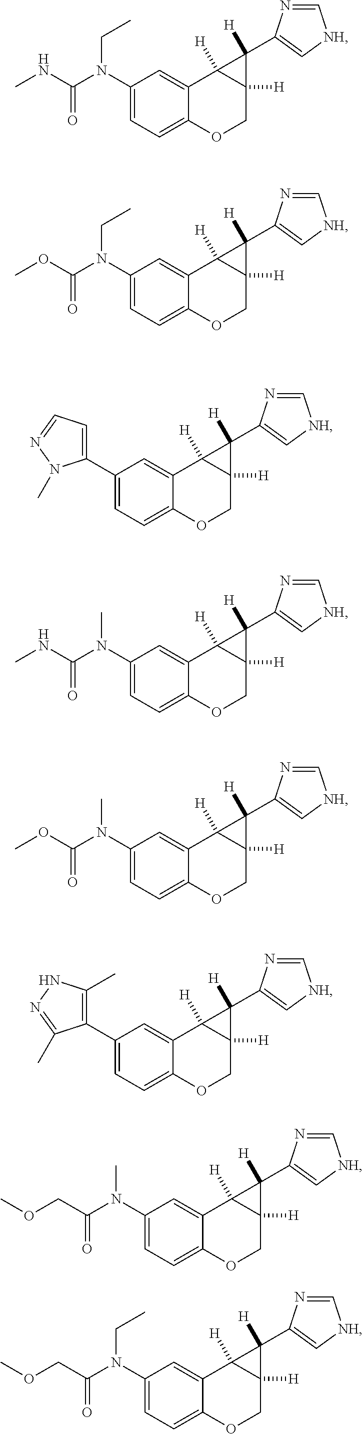

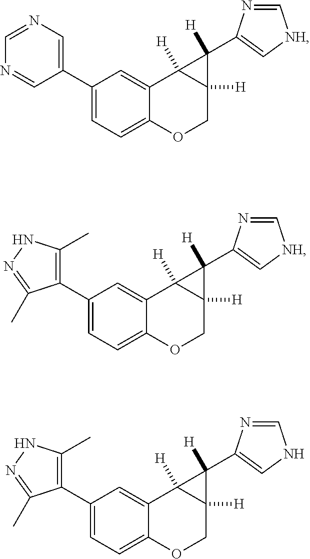

(wherein relative, but not absolute stereochemistry, is indicated) as well as the pharmaceutically acceptable prodrugs, salts, solvates or esters of each of these compounds.

In another embodiment the compound of Formula I or its pharmaceutically accept salt, solvate or ester thereof is present in its isolated and purified form.

One embodiment of the present invention is compounds that act as agonists of the α2C receptor. Alpha-2C receptor agonists can by used in the treatment or prevention of allergic rhinitis, congestion (including, but not limited to nasal congestion), migraine, congestive heart failure, chronic heart failure, cardiac ischemia, glaucoma, stress-induced urinary incontinence, attention deficit hyperactivity disorder, neuronal damage from ischemia and psychotic disorders. Further, alpha-2C receptor agonists can be useful in the treatment of pain (both chronic and acute), such as pain that is caused by inflammation, neuropathy, arthritis (including osteo and rheumatoid arthritis), diabetes (e.g., diabetes mellitus or diabetes insipidus) or pain of an unknown origin. Examples of neuropathic pain may include but not limited to; diabetic neuropathy, neuralgia of any etiology (e.g. post-herpetic, trigeminal), chemotherapy-induced neuropathy, HIV, lower back pain of neuropathic origin (e.g. sciatica), traumatic peripheral nerve injury of any etiology, central pain (e.g. post-stroke, thalamic, spinal nerve injury). Other pain that can be treated is nociceptive pain and pain that is visceral in origin or pain that is secondary to inflammation or nerve damage in other diseases or diseases of unknown origin. Further, alpha-2C receptor agonists can be useful in the treatment of symptoms of diabetes. Examples of symptoms of diabetes may include but are not limited to: hyperglycemia, hypertriglyceridemia, increased levels of blood insulin and hyperlipidemia.

A compound is defined to be an agonist of the alpha-2c receptor if the compound's efficacy at the α2C receptor is 30% Eₘₐₓ (GTPγS assay).

A further embodiment of the present invention are that act selectively, and preferably even specifically, as agonists of the α2C or the α2B/α2C (hereinafter referred to as α2C or α2B/2C) receptor subtypes in preference over the α2A receptor subtype and that act functionally selectively as agonists of the α2C or the α2B/2C receptor subtype in preference over the α2A receptor subtype possess desirable therapeutic properties associated with adrenergic receptors but without having one or more undesirable side effects such as changes in blood pressure or sedation. For the purposes of the present invention, a compound is defined to be a specific or at least functionally selective agonist of the α2C receptor subtype over the α2A receptor subtype if the compound's efficacy at the α2C receptor is 30% Eₘₐₓ (GTPγS assay) and its efficacy at the α2A receptor is ≦35% Eₘₐₓ, (GTPγS assay).

In another embodiment of the present invention the compound acts as an antagonist of the alpha-2C receptor. Alpha-2C receptor antagonists can be used in the treatment or prevention of disease states such as depression, schizophrenia, post tramautic stress disorder, Parkinson's disease, dementias (e.g., Alzheimer's disease and neuropathic disorders.

A compound is defined to be an antagonist of the alpha-2C receptor if the compounds's efficacy at the α2C receptor is <30% Eₘₐₓ (GTPγS assay) and the binding inhibition of at the α2C receptor (K) is <500 nM, preferably <200 nM, and most preferably <20 nM. In a further embodiment of the present invention, the α2C receptor subtype antagonists possess desirable therapeutic properties associated with the α2C adrenergic receptor but without having one or more undesirable side effects associated with α2A agonism. For the purposes of this invention, compounds that act as antagonists at the α2C receptor subtype preferably do not possess an efficacy at the α2A receptor of 35% Eₘₐₓ or more (GTPγS assay).

Alternatively, the present invention provides for a method for the treatment of congestion in a mammal in need thereof which comprises administering to a mammal an effective dose of at least one compound having adrenergic activity wherein said compound is a functionally selective agonist of the α2c receptor or the α2C/αB adrenergic receptor.

A further embodiment of the present invention is a method for the treatment of congestion in a mammal in need thereof which comprises administering to a mammal an effective dose of at least one compound having adrenergic activity wherein said compound is a functionally selective agonist of the α2C receptor or the α2C/αB adrenergic receptor, wherein the selective agonist of the α2c receptor or the α2C/αB adrenergic receptor has an efficacy that is greater than or equal to 30% Eₘₐₓ when assayed in the GTPγS assay and its efficacy at the α2A receptor is ≦35% Eₘₐₓ (GTPγS assay).

As used above, and throughout this disclosure, the following terms, unless otherwise indicated, shall be understood to have the following meanings:

“Patient” includes both human and animals.

“Mammal” means humans and other mammalian animals.

“alpha-2C modulator” or “α2C modulator” means that a compound has affinity for (or binds to) the α2C receptor which provokes a biological response (i.e., either an agonistic or antagonistic response).

“alpha-2C receptor agonist or “α2C receptor agonist” is a compound that has affinity for the α2C receptor and elicits a biological response that mimics the response observed by the endrogenous ligand (e.g., neurotransmitter) that binds to the same receptor.

“alpha-2C receptor antagonist or “α2C receptor antagonist” is a compound that has affinity for the α2C receptor and elicits a biological response that blocks or dampens the response observed by the endrogenous ligand (e.g., neurotransmitter) that binds to the same receptor.

“Congestion” refers to all type of congestion including, but not limited to, congestion associated with perennial allergic rhinitis, seasonal allergic rhinitis, non-allergic rhinitis, vasomotor rhinitis, rhinitis medicamentosa, sinusitis, acute rhinosinusitis, or chronic rhinosinusitis or when the congestion is caused by polyps or is associated with the common cold.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Aryl” means an aromatic monocyclic or multicyclic ring system, in which at least one of the multicyclic rings is an aryl ring, comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl. Non-limiting examples of aryl multicyclic ring systems include:

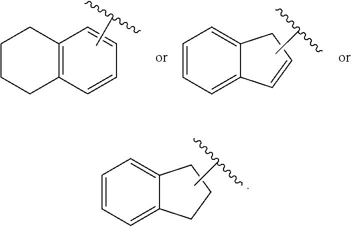

“Heteroaryl” means an aromatic monocyclic or multicyclic ring system, in which at least one of the multicyclic rings is aromatic, comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

Non-limiting examples of heteroaryl multicyclic ring systems include:

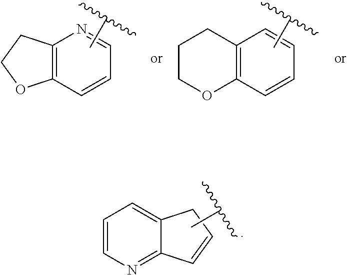

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Halogen” and “Halo” mean fluorine, chlorine, bromine, or iodine. Preferred are fluorine, chlorine or bromine, and more preferred are fluorine and chlorine.

“Ring system substituent” means a substituent attached to an aromatic or non-aromatic ring system which, for example, replaces an available hydrogen on the ring system. Ring system substituents may be the same or different, each being independently selected from the group consisting of aryl, heteroaryl, aralkyl, alkylaryl, heteroaralkyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, Y₁Y₂N—, Y₁Y₂N-alkyl-, Y₁Y₂NC(O)— and Y₁Y₂NSO₂—, wherein Y₁ and Y₂ may be the same or different and are independently selected from the group consisting of hydrogen, alkyl, aryl, and aralkyl.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protected moieties are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, imidazolidinyl, pyrazolidinyl, piperazinyl, morpholinyl, thiomorpholinyl, thiazolidinyl, 1,4-dioxanyl, tetrahydrofuranyl, tetrahydrothiophenyl, and the like.

Compounds of Formula I and salts, esters, solvates and prodrugs thereof, may exist in their tautomeric form (for example, as an amide or imino ether). All such tautomeric forms are contemplated herein as part of the present invention. Non-limiting examples of tautomeric forms that are part of this invention are as follows:

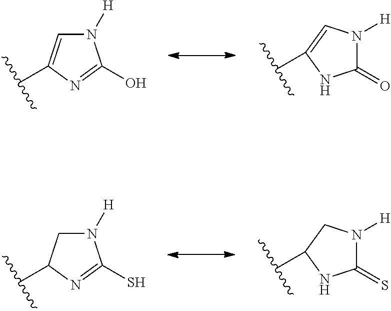

It should be noted that in saturated heterocyclyl containing systems of this invention, there are no hydroxyl, amino, or thiol groups on carbon atoms adjacent to a N, O or S atom. Thus, for example, in the ring:

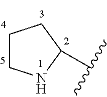

there is no —OH attached directly to carbons marked 2 and 5. It should also be noted that this definition does not preclude (═O), (═S), or (═N) substitutions, or their tautomeric forms, on C atoms adjacent to a N, O or S. Thus, for example, in the above ring, (═O) substitution on carbon 5, or its imino ether tautomer is allowed.

Non-limiting examples which illustrate the present invention are as follows:

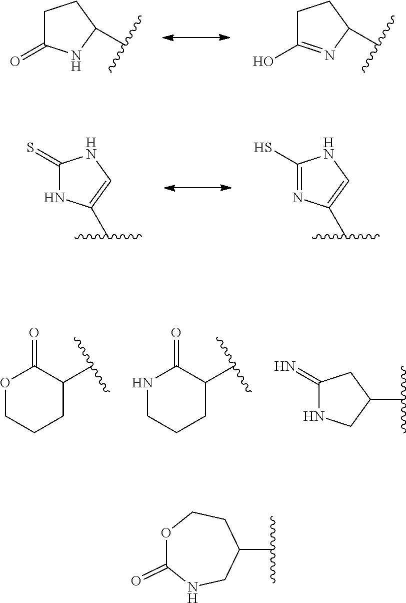

The following non-limiting examples serve to illustrate radicals not contemplated by the present invention:

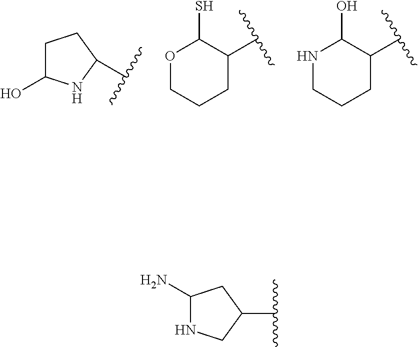

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Heterocyclylalkyl” means a heterocyclyl-alkyl group in which the heterocyclyl and the alkyl are as previously described. Preferred heterocyclylalkyls contain a lower alkyl group. Non-limiting examples of suitable heterocyclylalkyl groups include piperidylmethyl, piperidylethyl, pyrrolidylmethyl, morpholinyipropyl, piperazinylethyl, azindylmethyl, azetidylethyl, oxiranylpropyl and the like. The bond to the parent moiety is through the alkyl group.

“Heterocyclenyl” (or “heterocycloalkeneyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable monocyclic azaheterocyclenyl groups include 1,2,3,4-tetrahydropyridyl, 1,2-dihydropyridyl, 1,4-dihydropyridyl, 1,2,3,6-tetrahydropyridyl, 1,4,5,6-tetrahydropyrimidyl, 2-pyrrolinyl, 3-pyrrolinyl, 2-imidazolinyl, 2-pyrazolinyl, 2-oxazolinyl, 2-thiazolinyl, and the like. Non-limiting examples of suitable oxaheterocyclenyl groups include 3,4-dihydro-2H-pyran, dihydrofuranyl, fluorodihydrofuranyl, and the like. Non-limiting example of a suitable multicyclic oxaheterocyclenyl group is 7-oxabicyclo[2.2.1]heptenyl. Non-limiting examples of suitable monocyclic thiaheterocyclenyl rings include dihydrothiophenyl, dihydrothiopyranyl, and the like.

“Heterocyclenylalkyl” means a heterocyclenyl-alkyl group in which the heterocyclenyl and the alkyl are as previously described.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an organic acid group in which the —OH of the carboxyl group is replaced by some other substituent. Suitable non-limiting examples include H—C(O)—, alkyl-C(O)—, cycloalkyl-C(O)—, heterocyclyl-C(O)—, and heteroaryl-C(O)— groups in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” or “arylalkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Heteroarylalkoxy” means a heteroarylalkyl-O-group in which the heteroarylalkyl group is as previously described.

“Heterocyclylalkoxy” means a heterocyclylalkyl-O group in which the hetrocyclylalkyl group is as previously described.

“Heterocyclenylalkoxy” means a heterocyclenylalkyl-O group in which the heterocyclenylalkyl group is as previously described.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

It is noted that carbons of Formula I can be replaced with 1-3 silicon atoms, provided all valency requirements are satisfied.

The term “optionally substituted” means optional substitution with the specified groups, radicals or moieties.

The straight line [symbol] as a bond generally indicates a mixture of, or either of, the possible isomers, non-limiting example(s) include, containing (R)— and (S)-stereochemistry. For example,

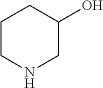

means containing both

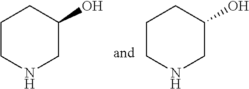

A dashed line ( [symbol] )represents an optional bond.

Lines drawn into the ring systems, such as, for example:

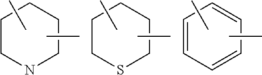

indicate that the indicated line (bond) may be attached to any of the substitutable ring atoms, non-limiting examples include carbon, nitrogen and sulfur ring atoms.

As well known in the art, a bond drawn from a particular atom wherein no moiety is depicted at the terminal end of the bond indicates a methyl group bound through that bond to the atom, unless stated otherwise. For example:

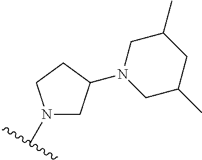

represents

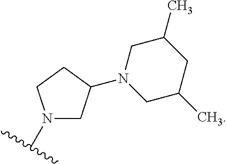

It should also be noted that any heteroatom with unsatisfied valences in the text, schemes, examples and Tables herein is assumed to have the hydrogen atom to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or formula, its definition on each occurrence is independent of its definition at every other occurrence.

Unless defined otherwise, all definitions for the variables follow the convention that the group to the right forms the point of attachment to the molecule; i.e., if a definition is arylalkyl, this means that the alkyl portion of the definition is attached to the molecule.

Further, all divalent variable are attached from left to right. For example when R¹ is —[C(R^{a})(R^{b})]_{q}N(R⁷)YR⁷ and Y is —C(═O)—, —C(═O)O— or —C(═O)NR⁷, then R¹ forms the group —[C(R^{a})(R^{b})]_{z}N(R⁷)—C(═O)—R⁷, —[C(R^{a})(R^{b})]_{q}N(R⁷)—C(═O)O—R⁷, or —[C(R^{a})(R^{b})]_{g}N(R⁷)—C(═O)N(R⁷)(R^{7′}).

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

For example, if a compound of Formula I or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Formula I contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I incorporates —NH— functional group, such as in a primary or secondary amine or in a nitrogen-containing heterocycle, such as imidazole or piperazine ring, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of illustrative solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

Metabolic conjugates, such as glucuronides and sulfates which can undergo reversible conversion to the compounds of Formula I are contemplated in the present invention.

“Effective amount” or “therapeutically effective amount” is meant to describe an amount of compound or a composition of the present invention effective in producing the desired therapeutic, ameliorative, inhibitory or preventative effect.

The terms “purified”, “in purified form” or “in isolated and purified form,” as used herein for a compound, refers to the physical state of said compound after being isolated from a synthetic process (e.g. from a reaction mixture), a natural source or a combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like) in sufficient purity to be characterizable by standard analytical techniques described herein or well known to the skilled artisan.

“Capsule” is meant to describe a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

“Tablet” is meant to describe a compressed or molded solid dosage form containing the active ingredients with suitable diluents. The tablet can be prepared by compression of mixtures or granulations obtained by wet granulation, dry granulation or by compaction.

“Oral gels” is meant to describe to the active ingredients dispersed or solubilized in a hydrophillic semi-solid matrix.

“Powders for constitution” refers to powder blends containing the active ingredients and suitable diluents which can be suspended in water or juices.

“Diluent” refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

“Disintegrants” refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

“Binders” refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

“Lubricant” is meant to describe a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d'l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

“Glidents” means materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

“Coloring agents” refers to excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

“Bioavailability” refers to the rate and extent to which the active drug ingredient or therapeutic moiety is absorbed into the systemic circulation from an administered dosage form as compared to a standard or control. Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of Formula I or may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons or sulfurs on various substituents, including enantiomeric forms (which may exist even in the absence of asymmetric carbons), rotameric forms, atropisomers, and diastereomeric forms, are contemplated within the scope of this invention. For example, if a compound of Formula I incorporates a double bond or a fused ring, both the cis- and trans-forms, as well as mixtures, are embraced within the scope of the invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Diasteromeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diasteromeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formulae Ia or Ib may be atropisomers (e.g., substituted biaryls) and are considered as part of this invention. Enantiomers can also be separated by use of chiral HPLC column.

Polymorphic forms of the compounds of Formula I, and of the salts, solvates and prodrugs of the compounds of Formula I, are intended to be included in the present invention.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples hereinbelow, by substituting an appropriate isotopically labelled reagent for a non-isotopically labelled reagent.

The compounds according to the invention have pharmacological properties; in particular, the compounds of Formula I can be useful as α2C adrenoreceptor modulators.

The compounds of Formula I can be purified to a degree suitable for use as a pharmaceutically active substance. That is, the compounds of Formula I can have a purity of 95 wt % or more (excluding adjuvants such as pharmaceutically acceptable carriers, solvents, etc., which are used in formulating the compound of Formula I into conventional forms, such as a pill, capsule, IV solution, etc. suitable for administration into a patient). The puriety can be 97 wt % or more, or, 99 wt. % or more. A purified compound of Formula I includes a single isomer having a purity, as discussed above, of 95 wt. % or more, 97 wt % or more or 99 wt. % or more, as discussed above.

A preferred dosage is about 0.001 to 500 mg/kg of body weight/day of the compound of Formula I. An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more therapeutic agents such as, for example, glucocorticosteroids, PDE-4 inhibitors, anti-muscarinic agents, cromolyn sodium, H₁ receptor antagonists, 5-HT₁ agonists, NSAIDs, angiotensin-converting enzyme inhibitors, angiotensin II receptor agonists, β-blockers, β-agonists (including both long and short acting), leukotriene antagonists, diuretics, aldosterone antagonists, ionotropic agents, natriuretic peptides, pain management/analgesic agents, anti-anxiety agents, anti-migraine agents, and therapeutic agents suitable for treating heart conditions, psychotic disorders, and glaucoma.

Suitable steroids include prednisolone, fluticasone (including all ester such as the propionate or furoate esters), triamcinolone, beclomethasone, mometasone (including any ester form such as mometasone furoate), budasamine, ciclesonide betamethasone, dexamethasone, prednisone, flunisolide, and cortisone.

Suitable PDE-4 inhibitors include roflumilast, theophylline, rolipram, piclamilast, cilomilast and CDP-840.

Suitable antiimuscarinic agents include ipratropium bromide and tiatropium bromide.

Suitable H₁ antagonists include astemizole, azatadine, azelastine, acrivastine, brompheniramine, cetirizine, chlorpheniramine, clemastine, cyclizine, carebastine, cyproheptadine, carbinoxamine, descarboethoxyloratidine, diphenhydramine, doxylamine, dimethindene, ebastine, epinastine, efletirizeine, fexofenadine, hydroxyzine, ketotifen, loratidine, levocabastine, meclizine, fexofenadine, hydroxyzine, ketotifen, loratadine, levocabastine, meclizine, mizolastine, mequitazine, mianserin, noberastine, norastemizole, picumast, pyrilamine, promethazine, terfenadine, tripelennamine, temelastine, trimeprazine or triprolidine.

Suitable anti-inflammatory agents include aspirin, diclofenac, diflunisal, etodolac, flurbiprofen, ibuprofen, indomethacin, ketoprofen, ketorolac, nabumetone, naproxen, oxaprozin, piroxicam, sulindac, and tolmetin.

Suitable aldosterone antagonists include spironolactone.

Suitable ionotropic agents include digitalis.

Suitable angiotensin II receptor agonists include irbesartan and losartan.

Suitable diuretics include spironolactone, methyclothiazide, bumetanide, torsemide, hydroflumethiazide, trichlormethiazide, hydroclorothiazide, triamterene, ethacrynic acid, methyclothiazide, hydrochlorothiazide, benzthiazide, hydrochlorothiazide, quinethazone, hydrochlorothiazide, chlorthalidone, furosemide, indapamide, hydroclorothiazide, triamterene, trichlormethiazide, hydrochlorothiazide, amiloride HCl, amiloride HCl, metolazone, trichlormethiazide, bendroflumethiazide, hydrochlorothiazide, polythiazide, hydroflumethiazide, chlorthalidone, and metolazone.

Suitable pain management/analgesic agents include Celecoxib, amitriptyline, ibuprofen, naproxen, gabapentin, tramadol, rofecoxib, oxycodone HCl, acetaminophenoxycodone HCl, carbamazepine, amitriptyline, diclofenac, diclofenac, etodolac, fenoprofen calcium, flurbiprofen, ibuprofen, indomethacin, ketoprofen, ketorolac tromethamine, mefenamic acid, meloxicam, nabumetone, naproxen, oxaprozin, piroxicam, sulindac, tolmetin sodium, valdecoxib, diclofenac/misoprostol, oxycontin, vicodin, darvocet, percocet, morphine sulfate, dilaudid, stadol, stadol NS, acetaminophen with codeine, acetaminophen with codeine #4, Lidoderm® patches, ziconotide, duloxetine, roboxetine, gabapentin and pregabalin.

Suitable β-blockers include acebutolol, atenolol, atenolol/chlorthalidone, betaxolol, bisoprolol fumarate, bisoprolol/HCTZ, labetolol, metoprolol tartrate, nadolol, pindolol, propranolol, propranolol/HCTZ, sotalol, and timolol.

Suitable β-agonists include dobutamine, ritodrine, salbutamol, levalbuterol, metaproternol, formoterol, fenoterol, bambuterol, brocaterol, clenbuterol, terbutaline, tulobuterol, epinephrine, isoprenalin, and hexoprenalin.

Suitable leucotriene antagonists include levamisole.

Suitable anti-migraine agents include rovatriptan succinate, naratriptan HCl, rizatriptan benzoate, sumatriptan succinate, zolmitriptan, almotriptan malate, methysergide maleate, dihydroergotamine mesylate, ergotamine tartrate, ergotamine tartrate/caffeine, Fioricet®, Fiorninal®, Depakene®, and Depakote®.

Suitable anti-anxiety and anti-depressant agents include amitriptyline HCl, bupropion HCl, citalopram hydrobromide, clomipramine HCl, desipramine, fluoxetine, fluvoxamine maleate, maprotiline HCl, mirtazapine, nefazodone HCl, nortriptyline, paroxetine HCl, protriptyline HCl, sertraline HCl, doxepin, and trimipramine maleate.

Suitable angiotensin converting enzyme inhibitors include Captopril, enalapril, enalapril/HCTZ, lisinopril, lisinopril/HCTZ, and Aceon®.

The pharmacological properties of the compounds of this invention may be confirmed by a number of pharmacological assays. The exemplified pharmacological assays which are described later have been carried out with the compounds according to the invention and their salts.

This invention is also directed to pharmaceutical compositions which comprise at least one compound of Formula I, or a pharmaceutically acceptable salt or solvate of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. When preparing a liquid preparation, the inclusion of one or more solubility enhancing components is excluded. Solubility enhancing components are described, for example, in U.S. Pat. No. 6,673,337 in column 2, line 50 to column 3, line 17 and in column 6, line 49 to line 31; U.S. Pat. No. 6,673,337 is expressly incorporated by reference. Specific solubility enhancing agents that are excluded in the liquid form preparations include metal carboxymethylcelluloses, metal carboxymethyl hydroxyethylcelloses, hydroxypropylmethyl celluloses derivative of these compounds, and cyclodextrins. As an example may be mentioned water or water-propylene glycol solutions for parenteral injection or addition of sweeteners and opacifiers for oral solutions, suspensions and emulsions. Liquid form preparations may also include solutions or suspensions for intranasal administration.

An aspect of this invention is that the pharmaceutical composition is in a solid dosage form comprising a compound of Formula I or a pharmaceutical acceptable salt, ester, solvate or prodrug thereof and a least one pharmaceutically acceptable carrier, adjuvant or vehicle.

Another aspect of this invention is a liquid, aqueous pharmaceutical composition is comprising a compound of Formula I or a pharmaceutical acceptable salt, ester, solvate or prodrug thereof and a least one pharmaceutically acceptable carrier, adjuvant or vehicle provided that the adjuvant is not a solubility enhancing component, such as those described in U.S. Pat. No. 6,673,337 (discussed above and herein incorporated by reference).

Another aspect of this invention is a liquid, aqueous pharmaceutical composition is comprising a compound of Formula I or a pharmaceutical acceptable salt, ester, solvate or prodrug thereof and a least one pharmaceutically acceptable carrier, adjuvant or vehicle wherein if a solubility enhancement component is present it is cyclodextrin.

Another aspect of this invention is a pharmaceutical formulation that is a nasal spray wherein the pH is equal to or less that about 6.5, more preferably between about 6.1 to 6.2.

Another aspect of this invention the formulation is a nasal spray wherein the adjuvants include a suspending agent (e.g., AVICEL (such as AVICIL RC-581, RC-591 and CL-611), which are microcrystalline cellulose and carboxymethylcellulose sodium; hydroxypropylmethyl cellulose; methyl cellulose; polyvinyl alcohol; or CARBOPOL) and a humectant (e.g., glycerin, propylene glycol; polyethylene glycol; povidone; or dextrose).

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt or solvate of said compound and a pharmaceutically acceptable carrier, vehicle or diluent.

Yet another aspect of this invention is a kit comprising an amount of at least one compound of Formula I, or a pharmaceutically acceptable salt or solvate of said compound and an amount of at least one therapeutic agent listed above, wherein the amounts of the two or more ingredients result in desired therapeutic effect.

In general, the compounds in the invention may be produced by a variety of processes know to those skilled in the art and by know processes analogous thereto. The invention disclosed herein is exemplified by the following preparations and examples which should not be construed to limit the scope of the disclosure. Alternative mechanistic pathways and analogous structures will be apparent to those skilled in the art. The practitioner is not limited to these methods.

One skilled in the art will recognize that one route will be optimized depending on the choice of appendage substituents. Additionally, one skilled in the art will recognize that in some cases the order of steps has to be controlled to avoid functional group incompatability.

The prepared compounds may be anyalyzed for their composition and purity as well as characterized by standard analytical techniques such as, for example, elemental anyalysis, NMR, mass spectroscopy and IR spectra.

One skilled in the art will recognize that reagents and solvents actually used may be selected from several reagents and solvents well known in the art to be effective equivalents. Hence, when a specific solvent or reagent is mentioned, it is meant to be an illustrative example of the conditions desirable for that particular reaction scheme and in the preparations and examples described below.

Where NMR data are presented, ¹H spectra were obtained on either a Varian VXR-400 (400 MHz, ¹H), Varian Gemini-300 (300 MHz), Varian Mercury VX-400 (400 MHz), or Bruker-Biospin AV-500 (500 MHz), and chemical shifts are reported as ppm with number of protons and multiplicities indicated parenthetically. Where LC/MS data are presented, analyses was performed using an Applied Biosystems API-100 mass spectrometer and C18 column, 10-95% CH₃CN—H₂O (with 0.05% TFA) gradient. The observed parent ion is given.

The following solvents and reagents may be referred to by their abbreviations in parenthesis:

Me=methyl; Et=ethyl; Pr=propyl; Bu=butyl; t-Bu=tert-butyl; Ph=phenyl, and Ac=acetyl
μl=microliters
AcOEt or EtOAc=ethyl acetate
AcOH or HOAc=acetic acid
ACN=acetonitrile
aq=aqueous
atm=atmosphere
Boc=tert-butoxycarbonyl
BINAP=2,2′-bis(diphenylphosphino)-1,1′-bisnaphthyl
cat=catalyst or catalytic
DEAD=diethylazodicarboxylate
DCM or CH₂Cl₂: dichloromethane:

### DMAP=4-Dimethylaminopyridine

DIPEA=diisopropylethylamine
DMF=dimethylformamide
DMS=dimethylsulfide
DMSO=dimethyl sulfoxide
EDCI or DEC=1-(3-dimethylaminopropyl)-3-ethylcarbodiimide
g=grams
h=hour
HOBt=1-hydroxybenzotriazole
LAH=lithium aluminum hydride
LCMS=liquid chromatography mass spectrometry
min=minute
mg=milligrams
mL=milliliters
mmol=millimoles
MeOH: methanol
MS=mass spectrometry
NBS=n-bromosuccimide

### NMO=N-methylmorpholine N-oxide

NMR=nuclear magnetic resonance spectroscopy
Pyr=pyridine
rac or (±)=racemic (mixture or enantiomers)
RT or rt=room temperature (ambient, about 25° C.)
sat=saturated
TBSCI=t-butyldimethylsilyl chloride
TBS=t-butyldimethyl silyl
TEA=triethylamine (Et₃N)
TEMPO=2,2,6,6-Tetramethylpiperidine-1-oxyl
TFA=trifluoroacetic acid
TPAP=tetrapropylammonium perruthenate
THF=tetrahydrofuran
TLC=thin layer chromatography
TMS=trimethylsilyl
Tos or Ts=p-toluenesulfonyl (tosyl)
Tol=toluene
TosMIC=Toluenesulfonylmethyl isocyanide
Tr=triphenylmethyl

### EXAMPLES

The compounds of this invention can be prepared through the general approach outlined in Schemes 1, 2 and 3. These schemes are being provided to illustrate the present invention. Group A is defined in these schemes in accordance with the definition in the invention; i.e., as an optionally substituted 5-membered heteroaryl, heterocyclyl or heterocyclenyl ring containing 1-3 heteroatoms. The depiction of A as an unsubstituted imidazole is not in any way to be considered a limitation of the invention scope. Likewise, the depiction of J¹-J³ in some of the schemes as CH should not to be seen as a limitation in the scope of the invention; it would be within the skill level of the practitioner to prepare compounds where the variables are other than CH by modifying the schemes in the appropriate manner. Group PG is an appropriate protecting group, the determination of which would be well within the skill level of one skilled in this art.

In Scheme 1, X represents O, S, CH₂, or N-PG, and Z is a substitution such as halogen, O-PG, NO₂ or N-PG. Ketone S1 can be reduced to the corresponding alcohol via one of numerous reagents known to those skilled in the art (such as NaBH₄, LAH or the like) and then eliminated to corresponding olefin (such as that catalyzed by treatment with acid or that facilitated by alcohol activation and elimination). Compound S2 can then undergo a cylcopropanation process, such as that faciliated by rhodium or copper or other related process. This step is followed by heterocycle A formation from the ester in S3. In the case where A=imidazole, the ester may be reduced to the aldehyde (or alternatively reduced to alcohol and oxidized to the aldehyde) followed by reaction with reagents such as TosMIC. Alternatively, other heterocycles may be formed from the ester, acid, or aldehyde following one of the many known literature methods to give compound S4. The heterocycle may be then protected (with groups such as trityl, BOC, or the like). Further reaction can be performed on the Z group to give the compound S5. For example, when Z=halogen or activated O, metal-catalyzed couplings may be performed. Alternatively when Z═NO₂, reduction and alkylation or acylation can provide alkyl anlines, amides, ureas, carbamates and the like. If appropriate, final deprotection of the heterocycle may then be performed.

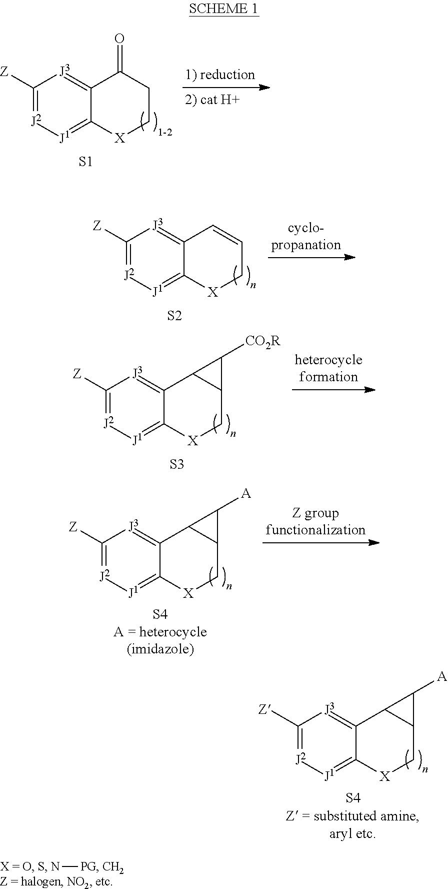

According to another embodiment, a five membered ring S7 may be synthesized as depicted in Scheme 2. Heterocycle S5, in which X═O (benzofuran), X═S (benzothiophene), or protected N (indole), may be cyclopropanated based on literature methods. For example, Angewandte Chemie Int. Ed. 2007, 46, 3889 details the asymmetric cyclopropanations of indole and indene compounds with Ir-salen complexes. Copper catalyzed cyclopropanations of indoles, benzofurans, and benzothiophenes have also been documented in the literature (for examples, see Tetrahedron Letters, 2004, 45, 4277; J. Org. Chem. 1977, 42, 3945; J. Chem. Soc, 1958, 1179 & 1183; and J. Chem. Soc. Perkin 1, 1990, 89). Compound S6 may then be further reacted to provide A as a heterocycle, such as imidazole, and Z′ as an appropriate functional group.

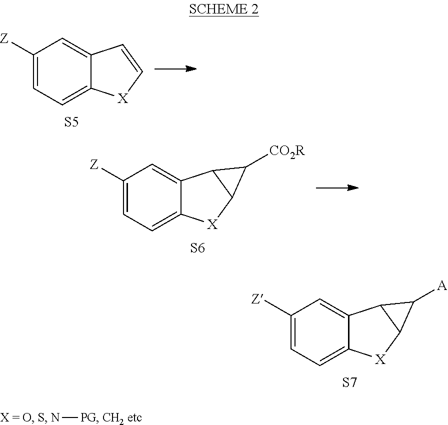

According to another embodiment, structures such as S4 may be synthesized as depicted in Scheme 3. In this case, cyclopropanation of S2 may be accomplished with a reagent such as S8, in which A′ is heterocycle, protected heterocycle, or a precursor to a heterocycle. (For an example of the synthesis of a similar diazocompound to S8, see the Journal of the American Chemical Society, 1958, 80, 6562).

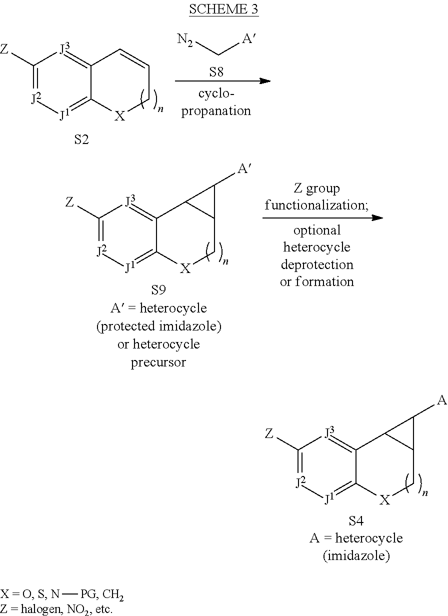

The starting materials and reagents described herein for preparing compounds described are either available from commercial suppliers such as Aldrich Chemical Co. (Wisconsin, USA) and Acros Organics Co. (New Jersey, USA) or are prepared by literature methods known to those skilled in the art.

Exemplary compounds are prepared as described in the examples below or from starting materials that are known in the art. These examples are being provided to further illustrate the present invention. They are for illustrative purposes only; the scope of the invention is not to be considered limited in any way thereby.

### Example 1

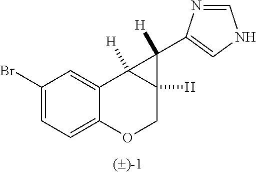

### Step 1

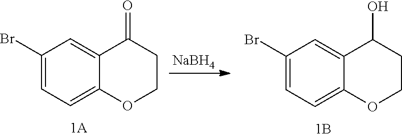

A mixture of 6-bromo-4-chromanone (1A, 120 g, 0.53 mol) in MeOH (600 mL) and DCM (400 mL) was cooled in an ice bath and treated with NaBH₄ (19.6 g, 0.53 mol) in several portions. The reaction was stirred at 0° C. for 0.5 h and then RT for 2 h. The reaction was then quenched with water and concentrated. The residue was taken up in EtOAc and sequentially washed with 1N aq HCl, brine, sat aq NaHCO₃ and brine. The organic layer was dried over Na₂SO₄, filtered and concentrated to provide crude 6-bromo-4-chromanol (1B, 122 g, ˜100%).

### Step 2

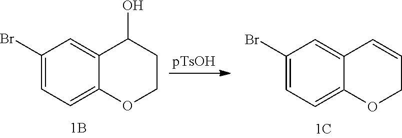

A mixture of 6-bromo-4-chromanol (1B, 50 g, 0.22 mol) in toluene (500 mL) was treated with catalytic pTsOH (4.18 g, 22 mmol) and refluxed for 3 h. The reaction was cooled to RT and then washed with sat aq NaHCO₃ and concentrated. Chromatography (0-20% EtOAc-hexanes) afforded 6-bromochromene 1C (43 g, 93%).

### Step 3

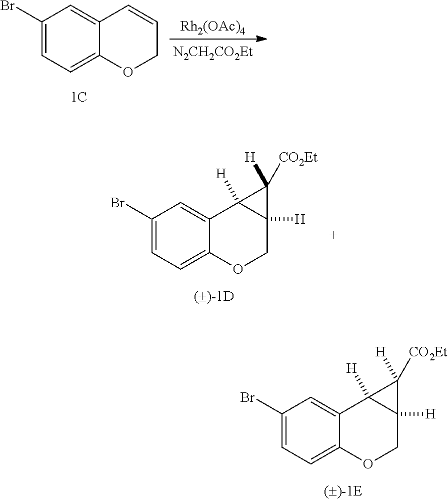

A slurry of the olefin 1C (42.2 g, 200 mmol) and Rh₂(OAc)₄ (8.84 g, 20 mmol) in DCM (1000 mL) was slowly treated with a solution of ethyl diazoacetate (N₂CH₂CO₂Et, 42 mL, 400 mmol) in DCM via syringe pump. Upon complete addition, the reaction was stirred for 1d at RT, filtered through a celite pad and concentrated. Chromatography (5-10% EtOAc-hexanes) afforded the less polar trans-isomer (±)-1D (31 g, 52%) and more polar cis-isomer (±)-1E (17.3 g, 29%).

### Step 4

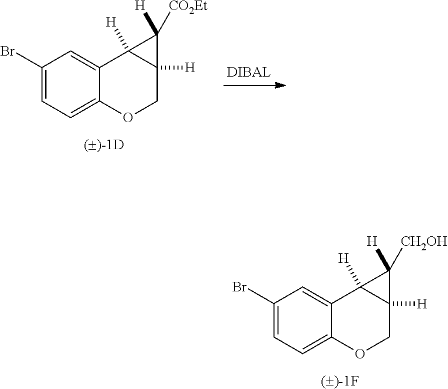

A solution of the trans-cycloproyl ester (±)-1D (30 g, 101 mmol) in THF at −78° C. was slowly treated with DIBAL (1.5M/toluene, 155 mL). The reaction was then slowly warmed to 0° C. and monitored by TLC. Upon complete consumption of starting material (±)-1D, the reaction was poured onto a mixture of sat aq NH₄Cl and ice, and extracted with EtOAc. The organic layer was washed with brine, dried over Na₂SO₄, filtered, and concentrated. Chromatography (EtOAc-hexanes) afforded the (±)-1F (25.4 g, 94%).

### Step 5

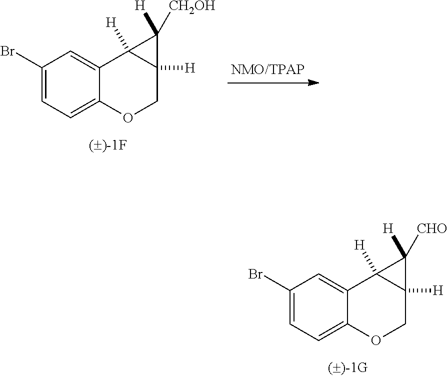

A solution of alcohol (±)-1F (25.4 g, 0.1 mol) in DCM (1 L) was treated with NMO (14 g, 0.12 mol) and then TPAP (1.76 g, 5 mmol). The reaction was stirred at RT for 1.5 h and then diluted with hexanes (800 mL). After stirring for 10 min, the mixture was filtered through a pad of celite and concentrated. Chromatography (EtOAc-hexanes) afforded the (±)-1G (18.8 g, 75%).

### Step 6

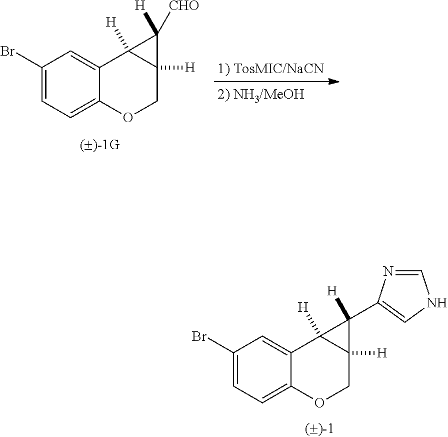

To a suspension of aldehyde (±)-1G (10.0 g, 40 mmol) and TosMIC (7.81 g, 40 mmol) in MeOH (200 mL) was added powdered NaCN (294 mg, 6 mmol) at 0° C. The reaction was stirred at 0° C. to RT over 3 h and then concentrated. The residue was taken up in NH₃-MeOH (7N, 200 mL) and then stirred in a sealed tube at 100° C. for 3d. The reaction was then concentrated, diluted with water and extracted with EtOAc. The organic layer was dried over Na₂SO₄, filtered, and concentrated. Chromatography (7N NH₃-MeOH in DCM) afforded the title compound (±)-1 (5.8 g, 50%).

### Example 2

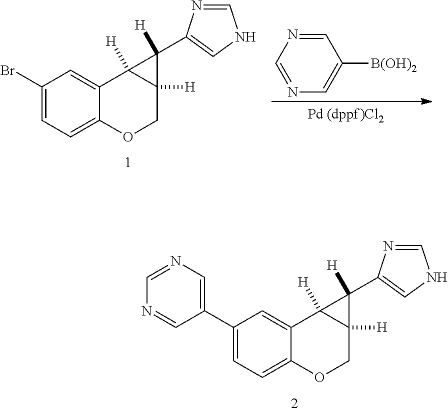

A mixture of compound (±)-1 (120 mg, 0.44 mmol), pyrimidine-5-boronic acid (77 mg, 0.62 mmol), Pd(dppf)Cl₂ (84 mg, 0.10 mmol), Na₂CO₃ (130 mg, 1.22 mmol) in 4:1 DME:water (6 mL) was microwaved at 120° C. for 16 min. The mixture was then partitioned between EtOAc and water. The organic layer was washed with brine, dried over Na₂SO₄, filtered and concentrated. Chromatography (7N NH₃-MeOH in DCM) provided compound (±)-2 (66 mg, 55%). LCMS m/z 291 (MH+).

In the following table, trans-cyclopropanes (±)-2A and (±)-2C−(±)-2F were prepared from compound (±)-1 or (±)-3A, based on the procedure outlined in Example 2. In the cases where compounds were prepared from (±)-3A, subsequent deprotection with TFA was performed similar to that found in Example 3 (Step 4). C is- cyclopropane (±)-2B was prepared from compound (±)-1E in a manner similar to that described in Example 1 (Steps 4-6: DIBAL reduction, NMO-TPAP oxidation, and imidazole formation) and Example 2 (Suzuki coupling).

| Cmpd | | LCMS |
|---|---|---|
| No. | Compound | (MH+) |
| (±)-2A | 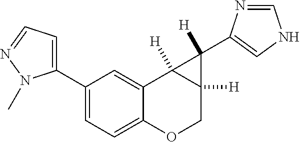 | 293 |
| (±)-2B | 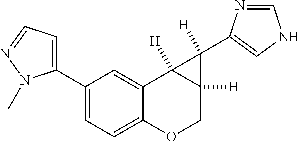 | 293 |
| (±)-2C | 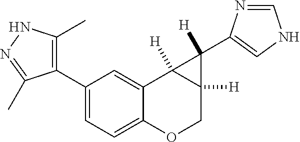 | 307 |
| (±)-2D | 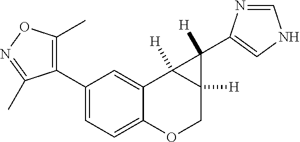 | 308 |
| (±)-2E | 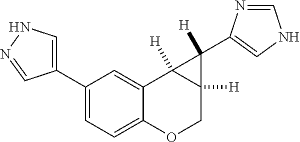 | 279 |
| (±)-2F | 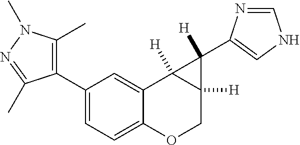 | 321 |

The pure single enanantiomers (2H and 21) of compound 2A were separated as described in the following procedure:

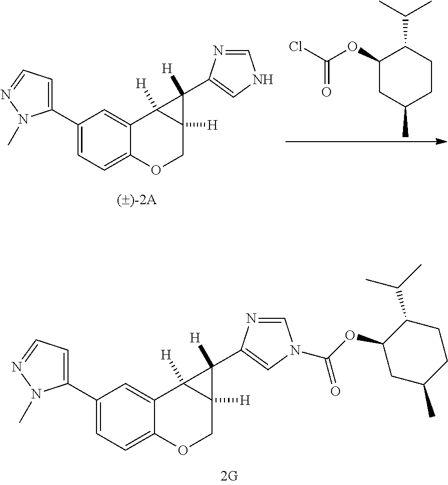

A solution of compound (±)-2A (10 mg, 0.034 mmol) in DCM was cooled to ° C. and treated sequentially with (−)-menthyl chloroformate (8.1 μL, 0.038 mmol) and TEA (5.3 μL, 0.038 mmol). The reaction mixture was stirred at room temperature until TLC indicated consumption of the starting material. The mixture was then quenched with water. The organic layer was separated, washed with brine, dried over Na₂SO₄ and concentrated. Chromatography provided compound 2G as a mixture of diastereomers (12 mg, 74%). The diastereomers were then separated on a preparative Chiralpak AD column with 20% isopropanol-hexanes to provide both diastereomer both in >95% ee.

The chiral auxiliary was cleaved from each diastereomer by treatment with 1:1 diethylamine-methanol to provide the single enantioisomers 2H and 21.

### Example 3

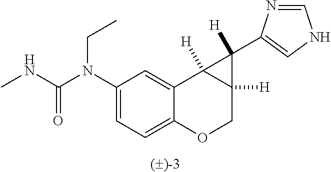

### Step 1

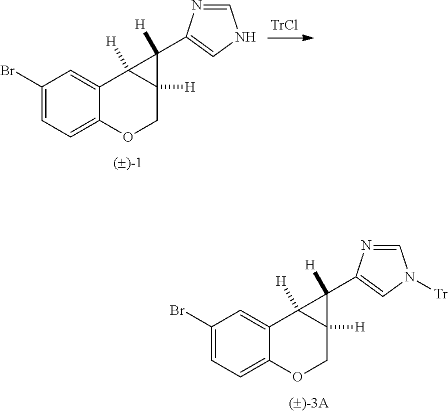

A mixture of compound (±)-1 (700 mg, 2.41 mmol) in THF (20 mL) was treated with TrCl (1.0 g, 3.61 mmol) and Et₃N (0.67 mL, 4.82 mmol) and then stirred at RT for 3d. The reaction was concentrated and chromatographed (EtOAc-hexanes) to provide (±)-3A.

### Step 2

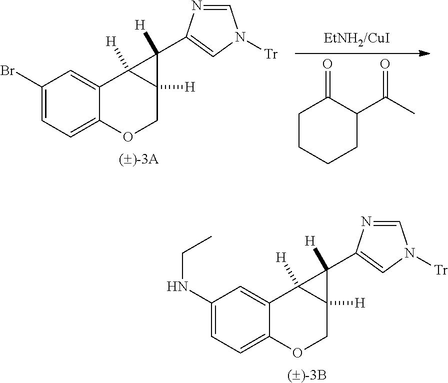

A mixture of compound (±)-3A (533 mg, 1.00 mmol), EtNH₂ (2.0N/THF, 1.5 mL), CuI (95 mg, 0.5 eq), K₃PO₄ (425 mg, 2 eq), and 2-acetylcyclohexanone (0.079 mL, 0.6 eq) in DMF (5 mL) was heated at 100° C. for 1d. The reaction was concentrated, diluted with water and extracted with DCM. The organic layer was dried over Na₂SO₄ and concentrated. Chromatography (0-5% of 7N NH₃-MeOH in DCM) provided (±)-3B as a light brown solid (300 mg).

### Step 3

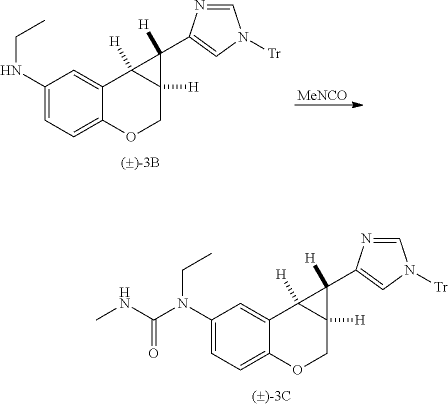

A mixture of the aniline (±)-3B (120 mg, 0.24 mmol) in DCM (5 mL) was treated with MeNCO (46 mg, 0.48 mmol) and stirred overnight at RT. The reaction was then quenched with MeOH, stirred 10 min, and concentrated. Chromatography (7N NH₃-MeOH in DCM) provided (±)-3C (109 mg, 82%).

### Step 4

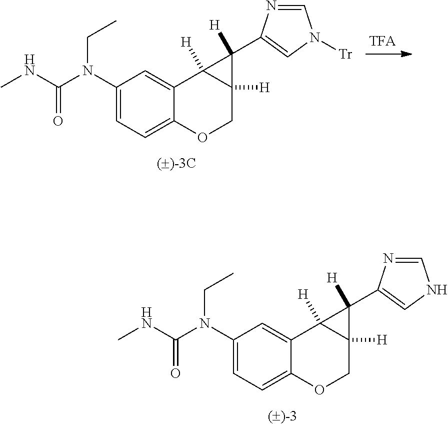

Compound (±)-3C (106 mg, 0.19 mmol) was taken up in DCM (10 mL) and treated with TFA (4 mL) and Et₃SiH (0.2 mL). The mixture was stirred at RT until TLC indicated complete consumption of (±)-3C. Chromatography (7N NH₃-MeOH in DCM) provided (±)-3 as a cream colored solid (54 mg, 91%). LCMS m/z 313 (MH+).

### Example 4

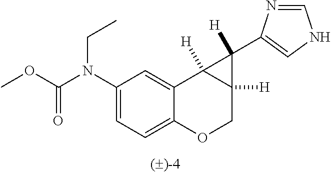

### Steps 1-2

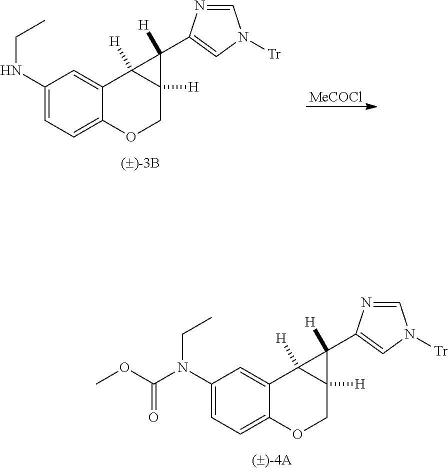

A mixture of the aniline (±)-3B (120 mg, 0.24 mmol) in DCM (4 mL) was treated with methyl chloroformate (28 μL, 0.36 mmol) and DIPEA (84 μL, 0.48 mmol). The reaction was stirred overnight at RT and then concentrated. Chromatography (7N NH₃-MeOH in DCM) provided (±)-4A (100 mg, 75%).

In a manner similar to that described in Example 3 (Step 4), (±)-4A was deprotected with TFA and Et₃SiH to provide the title compound (±)-4. LCMS m/z 314 (MH+).

### Example 5

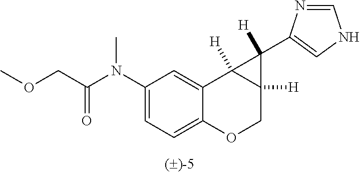

### Step 1

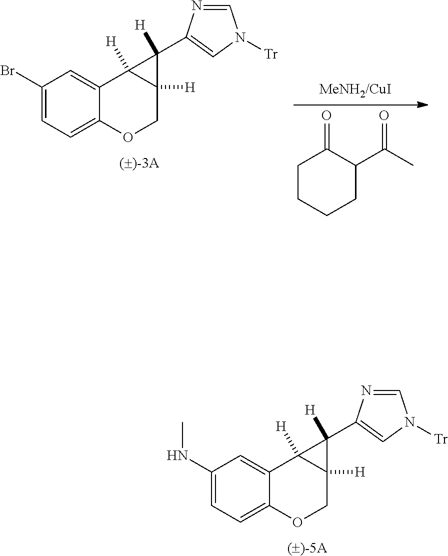

In a manner similar to that described in Example 3 (Step 2), compound (±)-3A was treated with a mixture of MeNH₂, CuI, K₃PO₄, and 2-acetylcyclohexanone in DMF to provide compound (±)-5A.

### Steps 2-3

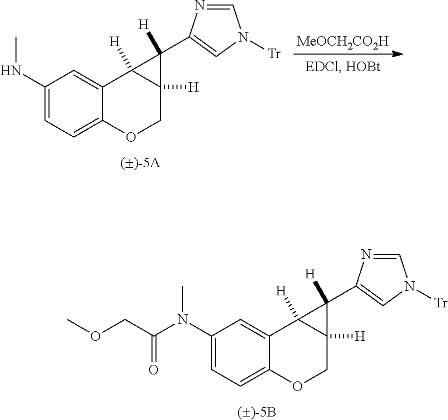

A mixture of the aniline (±)-5A (100 mg, 0.21 mmol) in 1:1 DCM:DMF (2.5 mL) was treated with methoxyacetic acid (24 mg, 1.3 eq), EDCI (61 mg, 1.5 eq), and HOBt (43 mg, 1.5 eq). The mixture was stirred overnight at RT, diluted with water and extracted with EtOAc. The organic layer was washed with water, washed with brine, dried over Na₂SO₄ and concentrated. Chromatography (7N NH₃-MeOH in DCM) provided (±)-5B as a solid (93 mg, 80%).

In a manner similar to that described in Example 3 (Step 4), (±)-5B was deprotected with TFA and Et₃SiH to provide the title compound (±)-5. LCMS m/z 314 (MH+).

### Example 6

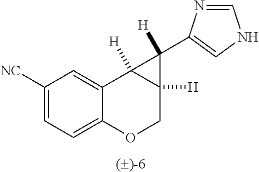

### Steps 1-2

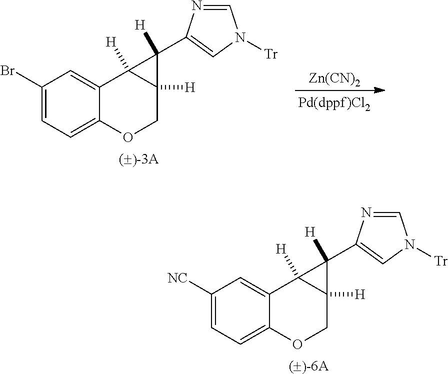

Based on a literature procedure (J. Med. Chem. 2007, 50, 1958), a mixture of compound (±)-3A (100 mg, 0.19 mmol), zinc cyanide (19 mg, 0.86 eq), zinc dust (10 mg, 0.82 eq), and Pd(dppf)Cl₂ (16 mg, 0.1 eq) in DMF (1.5 mL) was microwaved 160° C. for 20 min to provide (±)-6A.

In a manner similar to that described in Example 3 (Step 4), (±)-6A was deprotected with TFA and Et₃SiH to provide the title compound (±)-6. LCMS m/z 238 (MH+).

### Example 7

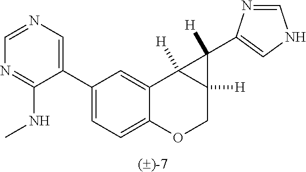

### Step 1

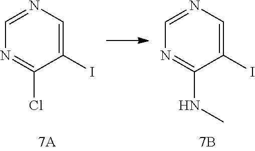

A solution of 4-chloro-5-iodopyrimidine 7A (2.03 g, 8.44 mmol) in 40% aqueous MeNH₂ solution was stirred at RT for 17 h. The mixture was then extracted with DCM (2×150 ml). The combined organic phase were dried concentrated to give 5-iodo-4-methylaminopyrimidine 7B (1.55 g, 78%) as an orange solid which was used for the next step without further purification.

### Steps 2-3

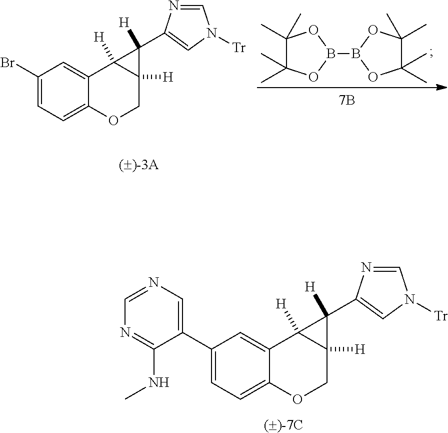

A mixture of compound (±)-3A (300 mg, 0.56 mmol), diboron pinacol ester (172 mg, 1.2 eq), Pd(dppf)Cl₂ (116 mg, 0.25 eq), KOAc (165 mg, 3 eq) in 1:1 DME-water (10 mL) in a sealed microwave vial was heated overnight in an oil bath at 120° C. The mixture was allowed to cool and then was treated with 5-iodo-4-methylaminopyrimidine 7B and K₂CO₃ (212 mg, 1.6 eq). The vial was resealed and then heated for 6 h in an oil bath at 120° C. The reaction was partitioned between EtOAc and water. The organic layer was washed with brine, filtered and concentrated. Chromatography (7N NH₃-MeOH in DCM) provided compound (±)-7C (160 mg, 51%). LCMS m/z 562 (MH+).

In a manner similar to that described in Example 3 (Step 4), (±)-7C was deprotected with TFA and Et₃SiH in DCM and then purified to provide the title compound (±)-7 as a light brown solid. LCMS m/z 320 (MH+).

### Example 8

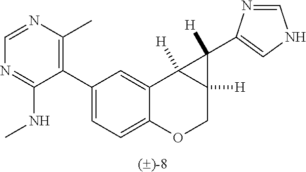

In a manner similar to that described in Example 7 (Step 1), 4-chloro-5-iodo-6-methylpyrimidine was treated with MeNH₂. The resulting product, 5-iodo-4-methylamino-6-methylpyrimidine, was then coupled with (±)-3A and deprotected with TFA in a manner similar to that described in Example 7 (Steps 2-3). The title compound (±)-8 was provided as a light brown solid. LCMS m/z 334 (MH+).

The following compounds were prepared following procedures similar to those exemplified in the examples above.

| | | LCMS |
|---|---|---|
| Cpd | Structure | (MH+) |
| (±)-100 | 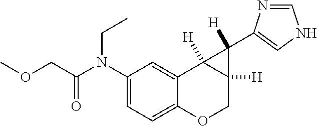 | 328 |
| (±)-101 | 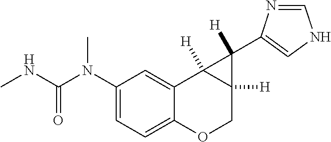 | 299 |
| (±)-102 | 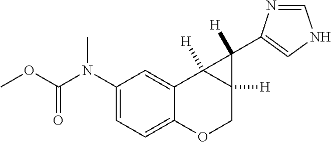 | 300 |
| (±)-103 | 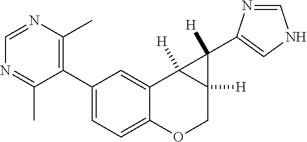 | 319 |

### Assay:

Efficacy agonist activity values (Emax, GTPγS assay) for α2A and α2C were determined by following the general procedure detailed by Umland et. al (“Receptor reserve analysis of the human α_{2c}-adrenoceptor using [³⁵S]GTPγS and cAMP functional assays” European Journal of Pharmacology 2001, 411, 211-221). For the purposes of the present invention, a compound is defined to be a specific or at least functionally selective agonist of the α2C receptor subtype if the compound's efficacy at the α2C receptor is 30% Emax (GTPγS assay) and its efficacy at the α2A receptor is ≦35% Emax (GTPγS assay). Additionally, for the purposes of this invention, a compound is defined to be an antagonist of the α2C receptor subtype if the compound's efficacy at the α2C receptor is <30% Emax (GTPγS assay) and the Kᵢ at the α2C receptor subtype was <500 nM, preferentially <200 nM, and most preferentially <20 nM.

The following compounds were evaluated to be active or functionally selective agonists of the α2C receptor subtype based on the previously defined definition: (±)-2A, (±)-2C, 2I, (±)-3, (±)-4, (±)-5, (±)-8, (±)-101, and (±)-102.

The following compounds were evaluated to be an antagonist of the α2C receptor subtype based on the previously defined definition (Kᵢ<200 nM): (±)-2, (±)-2d, (±)-2e, (±)-2f, (±)-7, and (±)-100.

While the present invention has been described with in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound represented by Formula I: 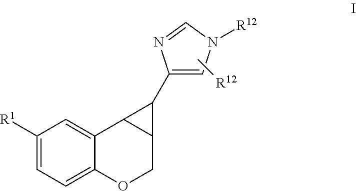
or a pharmaceutically acceptable salt, solvate, ester or prodrug thereof
wherein:
R¹ is selected from the group consisting of —[C(R^{a})(R^{b})]_{q}YR^{7′}, —[C(R^{a})(R^{b})]_{q}N(R⁷)YR^{7′}, —[C(R^{a})(R^{b})]ₚNR⁷R^{7′}, —[C(R^{a})(R^{b})]_{q}OYR⁷, —[C(R^{a})(R^{b})]_{q}N(YR⁷)(YR^{7′}), —[C(R^{a})(R^{b})]_{q}ON═CR⁷R^{7′} and —[C(R^{a})(R^{b})]_{q}CN;
Y is selected from the group consisting of a bond, —C(═O)—, —C(═O)NR⁷—, —C(═O)O—, —C(═O)—[C(R^{a})(R^{b})]ₙ—O—C(═O)—, —C(═O)N(R^{c})—O—, —C(═NR⁷)—, —C(═NOR⁷)—, —C(═NR⁷)NR⁷—, —C(═NR⁷)NR⁷⁰—, —C(═N—CN)—, —S(O)ₚ—, —SO₂NR⁷—, and —C(═S)NR⁷—;
wherein R^{a} and R^{b} are independently selected from the group consisting of H, alkyl, alkoxy, and halo, and
R^{c} is H or alkyl;
R⁷ and R^{7′} are each independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²;
R¹² is independently selected from the group consisting of H, halo, —OH, —CN, —NO₂, —N(R¹¹)₂, and —S(O)ₚR¹¹, and/or 1 or 2 (═O), and alkyl, alkoxy, alkenyl, alkenyloxy, alkynyl, cycloalkyl, cycloalkenyl, cycloalkoxy, aryl, aryloxy, arylalkyl, heteroaryl, heteroaryloxy, heteroarylalkyl, heterocyclyl, heterocyclenyl, heterocyclenyloxy, heterocyclylalkyl, heterocyclenylalkyl, arylalkoxy, heteroarylalkoxy, heterocyclylalkoxy, and heterocyclenylalkoxy groups,
p is independently 0, 1 or 2; and
q is independently an integer from 0-10;
provided that when
(a) R¹=—[C(R^{a})(R^{b})]_{q}YR^{7′}, q=0, and Y=—C(═O)O—, then R^{7′} cannot be H or alkyl;
(b) R¹=—[C(R^{a})(R¹³)]_{q}OYR^{7′}, q=0, and Y is a bond, then R^{7′} cannot be H or alkyl; and
(c) R¹=—[C(R^{a})(R^{b})]_{q}YR^{7′}, q=0, and Y is a bond, R^{7′} cannot be H.

2. (canceled)

3. The compound according to claim 1, wherein
R¹ is —[C(R^{a})(R^{b})]_{q}N(R⁷)YR^{7′}
Y is —C(═O)—, —C(═O)O— or —C(═O)NR⁷;
q is 0 or 1; and
or a pharmaceutically acceptable salt, ester or prodrug thereof.

4-5. (canceled)

6. The compound according to claim 1, which has Formula III 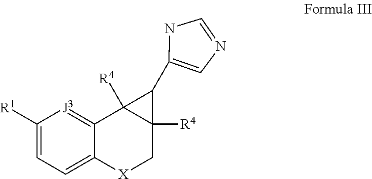
or a pharmaceutically acceptable salt, or ester thereof
wherein
J³ is —CH— or —N—;
R¹ is hydrogen, cyano, —N(R⁷)C(═O)OR^{7′}, —N(R⁷)C(═O)NH(R^{7′}), —N(R⁷)C(═O)R^{7′}, optionally substituted aryl, optionally substituted heteroaryl, optionally substituted heterocyclenyl, wherein said optionally substituted groups are optionally substituted one or more times by substituents independently selected from the group consisting of alkyl, halo, haloalkyl, alkoxy, haloalkoxy, cyano, nitro, amino, alkylamino, or dialkylamino;
R⁴ is independently H or alkyl;
R⁷ and R^{7′} are independently H, methyl, ethyl, propyl, cyclopropyl, wherein said methyl, ethyl, propyl or cyclopropyl groups are optionally substituted with one or more substituents selected from the group consisting of halogen, cyano of methoxy.

7. The compound according to claim 6, wherein X is —O—.

8. The compound according to claim 1, which is selected from the group consisting of: 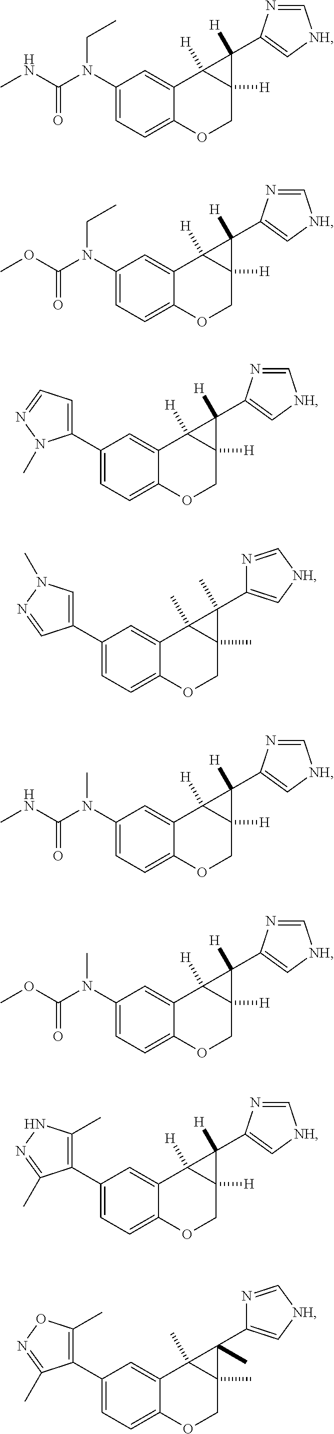 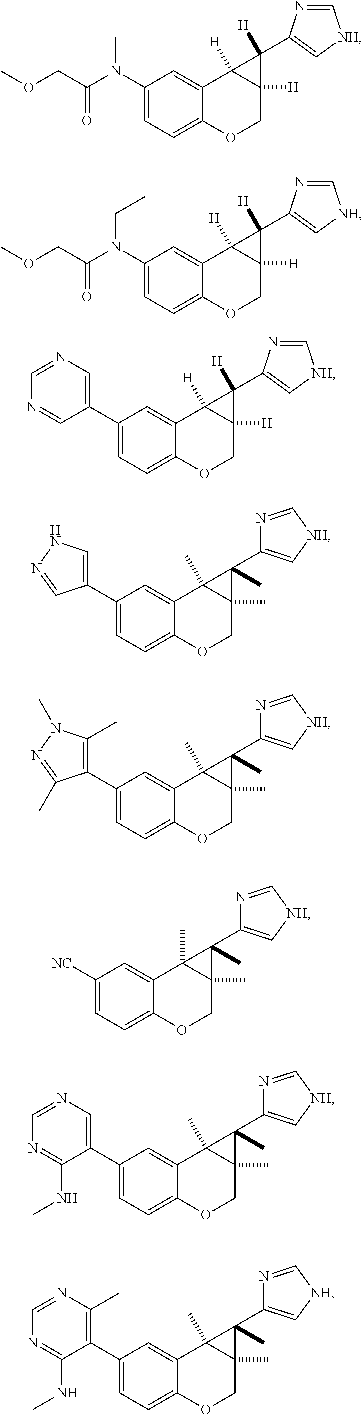 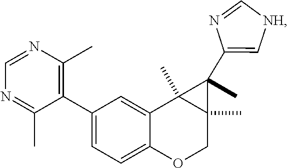
or a the pharmaceutically acceptable salt or ester of each of these compounds.

9. A pharmaceutical composition comprising at least one compound of claim 8 or a salt or ester thereof and at least one pharmaceutically acceptable carrier, adjuvant or vehicle, provided that when the composition is a liquid, aqueous composition one or more solubility enhancing components are excluded with the exception of cyclodextrin.

10. The pharmaceutical composition of claim 9, further comprising one or more additional therapeutic agents.

11. The pharmaceutical composition of claim 10, further comprising one or more additional therapeutic agents, wherein said additional therapeutic agents are selected from the group consisting of steroids, glucocorticosteroids, PDE-4 inhibitors, anti-muscarinic agents, muscle relaxants, cromolyn sodium, H₁ receptor antagonists, 5-HT₁ agonists, NSAIDs, angiotensin-converting enzyme inhibitors, angiotensin II receptor agonists, β-blockers, long and short acting β-agonists, leukotriene antagonists, diuretics, aldosterone antagonists, ionotropic agents, natriuretic peptides, pain management/analgesic agents, anti-anxiety agents, anti-migraine agents, sedatives, NMDA receptor antagonists, alpha-adrenergics not including alpha-1 receptor antagonists, anticonvulsants, tachykinin (NK) antagonists, COX-2 inhibitors, neuroleptics, vanilloid receptor agonists or antagonists, beta-adrenergics, local anaesthetic, corticosteroids, serotonin receptor agonists or antagonists, PDEV inhibitors, alpha-2-delta ligands, canabinoids and therapeutic agents suitable for treating heart conditions, psychotic disorders, or glaucoma.

12. A method for treating one or more conditions associated with α2C adrenergic receptors, comprising administering to a mammal in need of such treatment a compound of claim 1 or a pharmaceutically acceptable salt or solvate thereof.

13. The method of claim 12, wherein the conditions are selected from the group consisting of allergic rhinitis, congestion, pain, diarrhea, glaucoma, congestive heart failure, chronic heart failure, cardiac ischemia, manic disorders, depression, anxiety, migraine, stress-induced urinary incontinence, neuronal damage from ischemia, schizophrenia, attention deficit hyperactivity disorder, and symptoms of diabetes.

14. The method of claim 13, wherein the condition is congestion.

15. The method of claim 14, wherein the congestion is associated with perennial allergic rhinitis, seasonal allergic rhinitis, non-allergic rhinitis, vasomotor rhinitis, rhinitis medicamentosa, sinusitis, acute rhinosinusitis, or chronic rhinosinusitis.

16. The method of claim 14, wherein the congestion is caused by polyps or is associated with the common cold.

17. The method of claim 12, wherein the condition is pain.

18. The method of claim 17, wherein the pain is associated with neuropathy, inflammation, arthritis, or diabetis.

19. The method of claim 12, wherein the condition is Alzheimer's disease, depression, anxiety or Parkinson's disease.
